(19) United States
(12) Patent Application Publication
(10) Pub. No.: US 2022/0168325 A1
(43) Pub. Date: Jun. 2, 2022

(54) METHODS TO PROMOTE CEREBRAL BLOOD FLOW IN THE BRAIN

(71) Applicant: THE UNIVERSITY OF VERMONT, Burlington, VT (US)

(72) Inventors: Mark T. NELSON, Burlington, VT (US); Fabrice DABERTRAND, Hinesburg, VT (US); Osama F. HARRAZ, Burlington, VT (US); Masayo KOIDE, Richmond,, VT (US)

(21) Appl. No.: 17/598,008

(22) PCT Filed: Mar. 20, 2020
(86) PCT No.: PCT/US2020/023943

§ 371 Date: Sep. 24, 2021

Related U.S. Application Data
(60) Provisional application No. 62/823,378, filed on Mar. 25, 2019.

(51) Int. Cl. A61K 31/685; A61P 25/28

(52) U.S. Cl. CPC A61K 31/685; A61P 25/28

(57) ABSTRACT

The present application relates to methods for treating conditions characterized by reduced cerebral blood flow that include selecting a subject having a condition characterized by reduced cerebral blood flow. A therapeutic agent that increases the levels of PIP₂ is administered under conditions effective to treat the condition in the subject. Also disclosed are methods for treating CADASIL as well as methods for restoring cerebral blood flow and functional hyperemia.

This application claims the priority benefit of U.S. Provisional Patent Application Ser. No. 62/823,378, filed Mar. 25, 2019, which is hereby incorporated by reference in its entirety.

This invention was made with government support under grant numbers R01 HL136636, P01 HL-095488, R01 HL-121706, R37 DK-053832, 7UM-HL-1207704, and R01 HL-131181 awarded by National Institutes of Health. The government has certain rights in the invention.

### FIELD

The present application relates to methods to promote cerebral blood flow in the brain.

### BACKGROUND

Stroke and dementia, which show substantial co-morbidity and share multiple risk factors, rank among the most pressing health issues. Cerebral small vessel diseases (SVDs) have emerged as a central link between these two co-morbidities. Cerebral SVDs are a seemingly intractable ensemble of genetic and sporadic diseases that are major contributors to stroke and dementia (Chabriat et al., “CADASIL,” Lancet Neurol. 8(7):643-653 (2009)). SVDs of the brain, which progress silently for years before becoming clinically symptomatic, are responsible for more than 25% of ischemic strokes; they are also the leading cause of age-related cognitive decline and disability, accounting for more than 40% of dementia cases (Pantoni “Cerebral Small Vessel Disease: From Pathogenesis and Clinical Characteristics to Therapeutic Challenges,” Lancet Neurol. 9(7):689-701 (2010)). Hypertension, the leading cause of cardiovascular disease, is also the single greatest risk factor for SVDs. Indeed, a recent American Heart Association (AHA) Scientific Statement summarized evidence for structural, functional and cognitive consequences of hypertension, alone or in conjunction with ageing, that are consistent with the interpretation that hypertension is in fact a type of SVD (Iadecola et al., “Impact of Hypertension on Cognitive Function: A Scientific Statement From the American Heart Association,” Hypertension 68(6):e67-e94 (2016)). Despite the enormous impact of SVDs on human health, the disease processes and key biological mechanisms underlying these disorders remain largely unknown. However, accumulating experimental evidence suggests that functional or structural alterations in the cerebral microvasculature have early and deleterious consequences on the brain prior to or in association with the occurrence of the distinctive focal ischemic or hemorrhagic lesions characteristic of these diseases (Joutel et al., “Perturbations of the Cerebrovascular Matrisome: A Convergent Mechanism in Small Vessel Disease of the Brain?” J Cereb Blood Flow Metab. 36(1):143-157 (2016)). Notably, there are no specific treatments for these diseases (Chabriat., “CADASIL,” Lancet Neurol. 8(7):643-653 (2009)).

Cerebral blood flow (CBF) is exquisitely controlled to meet the ever-changing demands of active neurons. This activity-dependent blood delivery process (functional hyperemia) is rapidly and precisely controlled through a number of molecular mechanisms collectively termed ‘neurovascular coupling’ (NVC). Recent work provides unequivocal evidence that brain capillaries act as a neural activity-sensing network, showing that brain capillary endothelial cells (cECs) are capable of initiating an electrical (hyperpolarizing) signal in response to neural activity that rapidly propagates upstream to dilate feeding parenchymal arterioles (PAs) and locally increase blood flow. The mechanistic basis for this electrical signal has been further established, showing that extracellular K⁺—a byproduct of every neuronal action potential—is the critical mediator and the cEC strong inward rectifier K⁺ channel, Kir2.1, is the key molecular player.

Small vessel diseases—an ensemble of pathological processes that affect the microvasculature (arterioles, capillaries and venules) in the brain—are major contributors to stroke, disability, and cognitive decline that develop with aging and hypertension. CADASIL (Cerebral Autosomal Dominant Arteriopathy with Subcortical Infarcts and Leukoencephalopathy), caused by mutations in the NOTCH3 receptor, is the most common monogenic inherited form of SVD, and a model for more frequent sporadic forms. Transgenic mice expressing a mutant NOTCH3 (TgNotch3^{R169C}) found in CADASIL patients recapitulate salient clinical and histopathological hallmarks of the disease. Recent studies using this well-characterized model implicate altered extracellular matrix dynamics in this disease, showing that the matrix metalloproteinase inhibitor TIMP3 accumulates in NOTCH3 extracellular domain (NOTCH3^{ECD}) deposits surrounding vascular smooth muscle (SM) and pericytes. TIMP3 acts through inhibition of a disintegrin and metalloprotease 17 (ADAM17) to inhibit ectodomain shedding of the epidermal growth factor receptor (EGFR) ligand, heparin-binding EGF-like growth factor (HB-EGF), thereby suppressing EGFR pathway that normally regulates cerebral hemodynamics. The downregulation of the ADAM17/HB-EGF/EGFR signaling axis, causes signs of SVD, including impaired CBF control and functional and structural abnormalities in arterioles and capillaries. However, the mechanism(s) by which cerebral blood flow is compromised in SVD is not known.

It has recently been demonstrated that defective functional hyperemia (FH) is an early deficit in SVDs (Capone et al., “Mechanistic Insights into a TIMP3-Sensitive Pathway Constitutively Engaged in the Regulation of Cerebral Hemodynamics,” eLife 5:e17536 (2016)). In agreement with the observation of an early defect in FH in the CADASIL mouse model, a recent study demonstrated significant deficits in functional hyperemia in response to motor and visual stimulation at an early stage in CADASIL patients (mean age of 43 years), long before the occurrence of significant disability and cognitive decline typically associated with stroke and/or cerebral atrophy at the latest stage of the disease (Chabriat et al., “CADASIL,” Lancet Neurol. 8(7):643-53 (2009); Huneau et al., “Altered Dynamics of Neurovascular Coupling in CADASIL,” Ann. Clin. Transl. Neurol. (2018)). Consistent with the centrality of TIMP3 in this signaling cassette, genetic overexpression of TIMP3 recapitulates cerebrovascular deficits of the CADASIL model, and genetic reduction (haploinsufficiency) of TIMP3 in CADASIL model mice restores normal cerebrovascular function (Capone et al., “Reducing Timp3 or Vitronectin Ameliorates Disease Manifestations in CADASIL Mice.” Ann Neurol. 79(3):387-403 (2019)).

As noted above, there are currently no effective treatments or cures for small blood vessel diseases of the brain.

The present application is directed to overcoming these and other deficiencies in the art.

### SUMMARY

The present application relates to a method of treating a subject for a condition characterized by reduced cerebral blood flow. The method involves selecting a subject having a condition characterized by reduced cerebral blood flow and administering, to the selected subject, a therapeutic agent that increases the level of phosphatidylinositol 4,5-bisphosphate (PIP₂), under conditions effective to treat the condition characterized by reduced cerebral blood flow.

Another aspect of the present application relates to a method of treating cerebral autosomal-dominant arteriopathy with subcortical infarcts and leukoencephalopathy (CADASIL) in a subject. The method involves selecting a subject having cerebral autosomal-dominant arteriopathy with subcortical infarcts and leukoencephalopathy (CADASIL) and administering, to the selected subject, a therapeutic agent that increases the level of phosphatidylinositol 4,5-bisphosphate (PIP₂), under conditions effective to treat CADASIL in the selected subject.

A further aspect of the present application relates to a method of restoring cerebral blood flow in a subject. The method involves selecting a subject having a reduction in cerebral blood flow and administering, to the selected subject, a therapeutic agent that increases the level of phosphatidylinositol 4,5-bisphosphate (PIP₂), under conditions effective to restore cerebral blood flow in the selected subject.

Another aspect of the present application relates to a method of restoring functional hyperemia in a subject. The method involves selecting a subject having reduced functional hyperemia and administering, to the selected subject, a therapeutic agent that increases the level of phosphatidylinositol 4,5-bisphosphate (PIP₂), under conditions effective to restore functional hyperemia, in the selected subject.

Brain capillaries play a critical role in sensing neural activity and translating it into dynamic changes in cerebral blood flow to serve the metabolic needs of the brain. The molecular cornerstone of this mechanism is the capillary endothelial cell inward rectifier K⁺ (Kir2.1) channel, which is activated by neuronal activity—dependent increases in external K⁺ concentration, producing a propagating hyperpolarizing electrical signal that dilates upstream arterioles. As described herein, a key regulator of this process is identified, demonstrating that phosphatidylinositol 4,5-bisphosphate (PIP₂) is an intrinsic modulator of capillary Kir2.1-mediated signaling. It is further shown that PIP₂ depletion through activation of Gq protein-coupled receptors (GqPCRs) cripples capillary-to-arteriole signal transduction in vitro and in vivo, highlighting the potential regulatory linkage between GqPCR-dependent and electrical neurovascular-coupling mechanisms. These results collectively show that PIP₂ sets the gain of capillary-initiated electrical signaling by modulating Kir2.1 channels. Endothelial PIP₂ levels would therefore shape the extent of retrograde signaling and modulate cerebral blood flow.

Further, the data provided herein supports the concept that downregulation of inward rectifier K⁺ (Kir2.1) channels in capillary endothelial (cECs) cripples sensing of neural activity and is the major contributor to compromised functional hyperemia (FH) in CADASIL. It is demonstrated that pathogenic accumulation of TIMP3 disrupts capillary-to-arteriole signaling in CADASIL, and heparin binding EGF-like growth factor (HB-EGF) treatment restores capillary Kir2.1 channel activity and functional hyperemia. It has further been found that hypertension, the major driver of sporadic SVDs, also leads to age-dependent deterioration of this major FH mechanism. Evidence is provided that depletion of PIP₂, a minor inner leaflet lipid that binds the Kir2.1 channel and sustains its activity, is responsible for the deficit in FH. It is proposed that pathological process of SVD prevents normal activation of epidermal growth factor receptors (EGFRs), which leads to a loss of cEC PIP₂ that cripples retrograde electrical signaling and thus FH. Importantly, FH in CADASIL was rescued through exogenous application of PIP₂, suggesting a broad-spectrum approach for improving CBF control in disease. This work represents a novel therapeutic strategy for restoring local blood flow in the brain in various pathological settings.

### BRIEF DESCRIPTION OF THE DRAWINGS

FIGS. 1A-1C show Kir2.1 activity in capillary endothelial cells is sustained by an ATP-dependent mechanism. FIG. 1A shows representative traces of Kir2.1 currents in freshly isolated mouse capillary endothelial cells (cECs) bathed in 60 mM K⁺, recorded from 0 to 20 or 25 minutes using voltage-ramps (−140 to 40 mV). FIG. 1A, left, shows Kir2.1 currents recorded in the conventional whole-cell configuration (dialyzed cytoplasm, 0 mM Mg-ATP in the pipette solution). FIG. 1A, middle, shows Kir2.1 currents recorded in the perforated whole-cell configuration (intact cytoplasm). FIG. 1A, right, shows Kir2.1 currents recorded in the conventional whole-cell configuration in a cEC dialyzed with 1 mM Mg-ATP. FIG. 1B is summary data showing normalized Kir2.1 currents over time, recorded at −140 mV in the conventional whole-cell configuration (dialyzed cytoplasm) with 0 mM Mg-ATP in the pipette solution (black line), in the perforated whole-cell configuration (intact cytoplasm; gray line), and in the conventional whole-cell configuration (dialyzed cytoplasm) with 1 mM Mg-ATP in the pipette solution (grey line). Error bars represent SEM (n=6-9 per condition). FIG. 1C is summary data showing the concentration dependence and hydrolysis requirement for Mg-ATP—mediated Kir2.1 current preservation (duration, 15 minutes). Values are presented as means±SEM (*P<0.05, one-way ANOVA followed by Dunnett's multiple comparisons test; n=5-9 for Mg-ATP experiments and n=4 for ATP-γ-S experiments). % I/Iₘₐₓ is Kir2.1 current normalized to the maximum current (at t₀) and expressed as a percentage. n.s., not significant.

FIGS. 2A-2B show Ba²⁺ blocks inwardly rectifying currents in capillary endothelial cells. Inwardly rectifying current (black) evoked by a voltage ramp (300 ms, −140 to +40 mV) in capillary endothelial cells in conventional whole-cell (FIG. 2A; dialyzed cytoplasm) and perforated-patch (FIG. 2B; intact cytoplasm) configuration, and block by 100 μM Ba²⁺ (grey). Ba²⁺-sensitive currents (grey), obtained by subtraction of currents before and after the application of Ba²⁺, are shown below.

FIGS. 3A-3B show Mg-ATP-mediated maintenance of Kir2.1 currents is not prevented by inhibitors of PKC, PKG, or PKA. FIG. 3A is summary data showing that 1 mM Mg-ATP preserves Kir2.1 currents in dialyzed capillary endothelial cells (cECs) over a duration of 15 minutes compared with 0 mM Mg-ATP (˜36% decline), an effect that was unaltered by inhibitors of PKC (1 μM Gö6976; n=3), PKG (10 μM Rp-8-Br-PET-cGMPS; n=3), or PKA (1 μM H-89 dihydrochloride; n=3). FIG. 3B is summary data showing the absence of an effect of PKC, PKG, or PKA inhibitors on peak Kir2.1 current densities (at −140 mV) in cECs dialyzed with 1 mM Mg-ATP (n.s., not significant compared with control; one-way ANOVA followed by Dunnett's multiple comparisons test; n=5-17 per condition).

FIGS. 4A-4F show intracellular ATP and PIP₂ maintain Kir2.1 currents. FIG. 4A is a schematic diagram showing the ATP-dependent synthesis steps and pharmacological interventions in the pathway leading to the production of PIP₂. FIG. 4B shows representative traces of Kir2.1 currents recorded over 25 minutes in the conventional whole-cell configuration in a capillary endothelial cell (cEC) dialyzed with a pipette solution containing 0 mM Mg-ATP, with 10 μM of the soluble form of PIP₂ diC8-PIP₂. FIG. 4C shows changes in Kir2.1 currents over time, recorded in the conventional whole-cell configuration in cECs dialyzed with a pipette solution containing 0 mM Mg-ATP, with or without (control) 10 μM diC8-PIP₂. Currents obtained at 15 minutes are expressed as a percentage relative to those at t₀ (time of acquisition of whole-cell electrical access). Data are presented as means±SEM (**P<0.01 unpaired Student's t test, n=9-10). FIG. 4D shows individual-value plots of peak inward currents in cECs, measured at −140 mV (at t₀) using the perforated whole-cell configuration (intact cytoplasm) or conventional whole-cell configuration in cECs dialyzed with a pipette solution containing 0 mM Mg-ATP, 1 mM Mg-ATP, or 0 mM Mg-ATP+10 μM diC8-PIP₂. Whole-cell capacitance averaged 8.6 pF. There were no significant differences among groups (one-way ANOVA followed by Dunnett's multiple comparisons test, n=19-57). FIG. 4E shows representative traces of Kir2.1 currents in a cEC with intact cytoplasm (perforated configuration) before (control) and 15 minutes after incubation with the PIP5K inhibitor UNC3230 (100 nM).

FIG. 4F shows individual-value plots showing effects of the PIP₂ synthesis inhibitors PIK93 (PI4K inhibitor, 300 nM), PAO (PI4K inhibitor, 10 μM), and UNC3230 (PIP5K inhibitor, 100 nM) on Kir2.1 currents in cytoplasm-intact cECs. Inhibitors were bath-applied immediately after t₀, and currents were compared before and 15 min after incubation (*P<0.05, one-way ANOVA followed by Dunnett's multiple comparisons test).

FIGS. 5A-5F show PGE2 inhibits Kir2.1 current in cECs by reducing PIP₂ levels. FIG. 5A is a schematic depiction of PIP₂ depletion by GqPCR activation through PLC-mediated hydrolysis to IP3 and diacylglycerol (DAG). FIG. 5B shows representative traces of Kir2.1 currents in a dialyzed capillary endothelial cell (cEC; 0 mM Mg-ATP) at different time points after addition of PGE2 (2 μM) showing accelerated current decline following GqPCR activation. FIG. 5C shows individual-value plots showing the enhancement of cEC Kir2.1 current decline by bath-applied PGE2 (2 μM; n=5) compared with time controls (no PGE2; n=9; #P<0.05 unpaired Student's t test) and rescue by 10 μM diC8-PIP₂ (dialyzed; n=3) or 10 μM U73122 (bath-applied; n=3). Currents were recorded upon access to the cell interior (t₀) and after 15 minutes in cECs dialyzed with 0 mM Mg-ATP-pipette solution. Changes in Kir2.1 currents were calculated as values obtained at 15 minutes relative to those at t₀, expressed as a percentage. Individual data points are shown together with means (long horizontal lines) and SEM (error bars) (**P<0.01, one-way ANOVA followed by Dunnett's multiple comparisons test). FIG. 5D shows representative current traces showing no effect of the PKC inhibitor Gö6976 (1 μM; bath-applied) or rapid cytosolic Ca²⁺ chelation with BAPTA (5.4 mM; dialyzed) on the PGE2-induced decline of Kir2.1 currents in cECs dialyzed with 0 mM Mg-ATP. FIG. 5E shows individual-value plots showing the effects of the prostanoid receptor blockers AH6809 (10 μM, n=3) and SC51322 (1 μM, n=3) on the enhancement of Kir2.1 current decline in cECs by PGE2, recorded under cytoplasm-intact conditions over a 15-minute period. Changes in Kir2.1 currents were calculated as values obtained at 15 minutes relative to those at t₀, expressed as a percentage. Individual data points are shown together with means (long horizontal lines) and SEM (error bars) (####P<0.0001, unpaired Student's t test, n=6; **P<0.01, one-way ANOVA followed by Dunnett's multiple comparisons test). FIG. 5F shows the effects of GqPCR agonists on normalized Kir2.1 current decline in cECs. Kir2.1 currents were recorded in the perforated patch configuration over 15 minutes in the absence (control) or presence of bath-applied PGE2 (2 μM), carbachol (CCh, 10 μM), oxotremorine M (Oxo-M, 10 μM), SLIGRL-NH2 (5 μM), or ATP (30 μM). Horizontal lines indicate means (n=4-6 each).

FIGS. 6A-6C show changes in PIP₂ levels, rather than its metabolites, IP3 and diacylglycerol, underlie the inhibitory effect of PGE2 on Kir2.1 channels. FIG. 6A is a schematic illustration showing that GqPCR activation evokes PIP₂ hydrolysis to IP3, which activates IP3 receptors (IP3Rs) and Ca²⁺ release from intracellular stores, and diacylglycerol (DAG), which activates PKC. FIG. 6B is a bar graph showing the suppressive effect of PGE2 (2 μM; bath applied for 15 minutes) on Kir2.1 currents, recorded in the conventional whole-cell configuration in capillary endothelial cells dialyzed with 0 mM Mg-ATP while signaling cascades downstream of PIP₂ hydrolysis are intact (n=6). FIG. 6C shows lack of an effect of simultaneously blocking PKC (1 μM Gö6976, bath-applied) and IP3R/Ca²⁺ (30 μM CPA, bath-applied; 5.4 mM BAPTA dialyzed) on PGE2-mediated suppression of Kir2.1 currents (n.s., not significant, unpaired Student's t test vs. intact PIP₂ metabolite signaling in FIG. 6B). Recording conditions are as in FIG. 6B (n=15). FIG. 6C, top right, is a schematic depiction of experimental paradigm, showing pharmacological interdiction points in the signaling cascades downstream of PIP₂ breakdown (red Xs) and pharmacological interventions. FIG. 6C, bottom, shows the current-voltage relationship illustrating Kir2.1 currents in the absence (n=11) or presence (n=15) of PGE2 (2 μM, 15-minute incubation).

FIGS. 7A-7C show GqPCR stimulation cripples capillary-to-arteriole electrical signaling. FIG. 7A is a representative diameter recording showing the time course of the inhibitory effect of bath-applied PGE2 (1 μM) on upstream arteriolar dilations induced by successive focal applications of 10 mM K⁺ (18 s, 5 psi) onto capillary segments in a capillary-parenchymal arteriole (CaPA) preparation (schematic, right inset). Relations above the trace indicate the processes occurring in the presence of PGE2 [dissociation of PIP₂ from Kir2.1 and hydrolysis of PIP₂ to diacylglycerol (DAG)] and washout (reassociation of PIP₂ with Kir2.1). FIG. 7B is summary data for experiment in FIG. 7A, showing K⁺-induced dilations from five CaPA preparations (n=5 mice), calculated as a percentage of maximal diameter responses (obtained in 0 mM Ca²⁺ at the conclusion of each experiment). Results were best fit as a plateau (lag phase) followed by one-phase exponential decay (R²=0.85). Lag phase (X₀)≈18 minutes; time constant of the postplateau exponential decay phase (τ_{decay})≈4 minutes. FIG. 7C shows Kir2.1 current decline following application of 2 μM PGE2 onto capillary endothelial cells (cECs) at t₀ (i.e., upon achieving electrical access), recorded in the perforated-patch (intact cytoplasm) configuration. Time constant of the exponential decay phase (τ_{decay})≈12 minutes (one-phase exponential decay, R²=0.85). Note the absence of a lag phase for Kir2.1 current decline. At X₀ (18 minutes), corresponding to the lag phase before detecting a decrease in dilatory response (in FIG. 7B), Kir2.1 current had declined by ˜53%.

FIGS. 8A-8D show muscarinic receptor stimulation cripples capillary-to-arteriole electrical signaling. FIG. 8A is a representative diameter recording of an arteriole in the ex vivo capillary-parenchymal arteriole (CaPA) preparation showing a gradual reduction in K⁺-induced upstream arteriolar vasodilation in the presence of bath-applied carbachol (CCh, 10 μM). Dilations were induced by pressure ejection (18 s, 5 psi) of 10 mM K⁺ onto capillaries (indicated by dots). FIG. 8B is summary data for the experiment in FIG. 8A showing best fit of results from four CaPA preparations (n=4 mice) as a lag phase (X₀≈12 minutes) followed by one-phase exponential decay (τ_{decay}≈13 minutes, R²=0.83). FIG. 8C shows a representative trace of Kir2.1 currents recorded over 35 minutes in a capillary endothelial cell (cEC) using the perforated whole-cell configuration (intact cytoplasm) at different time points after the application of CCh (10 μM). FIG. 8D is summary data for Kir2.1 current decline following application of 10 μM CCh onto cECs at t₀ (i.e., upon achieving electrical access to the cell), recorded in the perforated-patch configuration (n=4 cECs from four mice). Time constant of the exponential decay phase (τ_{decay}) 8 minutes (one-phase exponential decay, R²=0.94). At X₀ (12 minutes), corresponding to the lag phase in FIG. 8B, Kir2.1 current had declined by ˜58%.

FIGS. 9A-9E show activation of cEC muscarinic receptors attenuates K⁺-induced increases in capillary red blood cells (RBC) flux in vivo. FIG. 9A is a 3D projection depicting the positioning of a pipette containing artificial cerebrospinal fluid with 10 mM K⁺ and red fluorescence tagged (TRITC)-dextran adjacent to a brain cortex capillary in vivo. Green fluorescence tagged (FITC)-dextran is circulating in blood plasma. FIG. 9B, top, shows raw capillary line-scan data showing RBCs (black streaks) in plasma; the x axis is time and they axis is scanned capillary distance (d). FIG. 9B, middle and bottom, shows line scans at baseline and in response to ejection of K⁺ (10 mM) onto the target capillary in a control (saline-injected) mouse and a mouse injected with carbachol (CCh, 0.6 μg/kg). Mice were systemically administered saline or CCh 20 min before applying 10 mM K⁺ by pressure ejection. At the conclusion of experiments, 0 mM Ca^{2+/}200 μM diltiazem was applied to the brain surface to evoke near-maximal arteriolar dilation and increase blood flow to the capillary bed to provide a frame of reference for the modest and sub-maximal increases in basal RBC flux sometimes observed in CCh-injected mice. Each line scan spans 1 s. FIG. 9C shows the time course of capillary RBC flux corresponding to the experiments in FIG. 9B in response to ejection of K⁺ (10 mM) onto a capillary in a control (saline-injected) and a CCh-treated mouse, showing elimination of K⁺-induced dilation by activation of capillary endothelial cell muscarinic receptors. FIG. 9D shows changes in K⁺ (10 mM)-induced capillary RBC flux over 30 min in saline- and CCh-treated mice (n=6-7). Changes in flux at 10, 20, and 30 minutes were normalized to their respective baseline values. FIG. 9E is summary data showing the percentage change in RBC flux in response to K⁺ (10 mM) 20 minutes after saline (n=5) or CCh (n=7) treatment (**P<0.01, unpaired Student's t test).

FIGS. 10A-10B show effects of in vivo muscarinic receptor stimulation on baseline capillary RBC flux and parenchymal arteriolar diameter. FIG. 10A shows baseline capillary RBC flux (before application of 10 mM K⁺) at different time points (zero, 10, 20, and 30 minutes) in mice treated with saline (n=6) or carbachol (CCh, n=8), showing no differences between groups (one-way ANOVA followed by Dunnett's multiple comparisons test). Maximum RBC flux was obtained at the end of the experiment by surface application of artificial cerebrospinal fluid containing 0 mM Ca²⁺ (0 Ca²⁺) and supplemented with 200 μM diltiazem (dilt) onto the cranial surface. FIG. 10B is summary data showing diameters of parenchymal arterioles upstream of the stimulated capillary segments monitored after treatment with CCh or saline. Data were obtained 20 min after systemic administration of CCh or saline. Maximum dilation was obtained at the end of the experiment by surface application of artificial cerebrospinal fluid containing 0 mM Ca²⁺ (0 Ca²⁺) supplemented with 200 μM diltiazem (dilt) (*P<0.05, two-way ANOVA with Tukey's multiple comparisons test, n=4 mice per group).

FIGS. 11A-11B show GqPCR activation inhibits Kir2.1 channel in a PIP₂-dependent manner. FIG. 11A is a schematic illustration showing that PIP₂ tonically sustains Kir2.1 channel activity under basal condition (no GqPCR activation), ensuring effective electrical capillary-to-arteriole signaling. In contrast, FIG. 11B shows GqPCR activation with an agonist (A) activates PLC, which hydrolyzes PIP₂ into the metabolites, diacylglycerol (DAG) and IP3. The decline in PIP₂ levels suppresses Kir2.1 channel activity and deactivates electrical signaling independent of PIP₂ metabolite-mediated signaling.

FIGS. 12A-12B show inclusion of GTP in the pipette solution does not alter Kir2.1 channel activity in capillary endothelial cells. FIG. 12A is a bar graph of averaged peak inward currents in capillary endothelial cells (cECs), measured at −140 mV (at t₀) using the conventional whole-cell configuration in cECs dialyzed with a pipette solution containing 100 μM GTP alone (black) or together with 1 mM Mg-ATP (gray). Averages were similar between the two groups (unpaired Student's t test, P=0.6, n=4 cECs per group). FIG. 12B is summary data showing normalized Kir2.1 currents over time, recorded at −140 mV in the conventional whole-cell configuration with 100 μM GTP and 0 mM Mg-ATP in the pipette solution (black solid line with error bars) or dialyzed with 100 μM GTP and 1 mM Mg-ATP (gray solid line with error bars). Error bars represent SEM (n=3 cECs per condition). Dotted lines represent average changes in current behavior in cECs dialyzed with 0 μM GTP in the absence (gray dotted line) or presence (pink dotted line) of Mg-ATP (1 mM), as depicted in FIG. 12B.

FIGS. 13A-13D show heparin-binding epidermal growth factor-like growth factor (HB-EGF) restored whisker stimulation-induced functional hyperemia in CADASIL model mice. FIG. 13A is representative traces of change in cerebral blood flow (CBF) during whisker stimulation in CADASIL model (TgNotch3^{R169C}) and control (TgNotch3^{WT}) mice. The traces in gray line show whisker stimulation-induced CBF changes after the treatment with Kir channel blocker, Ba²⁺. FIG. 13B is the summary showing that whisker stimulation-induced functional hyperemia was significantly attenuated in CADASIL model mice compare to control (TgWT) mice. FIG. 13C is the example traces of whisker-stimulation-induced CBF change before and after the treatment of Kir channel blocker, Ba²⁺, in the presence of HB-EGF. FIG. 13D is the summary showing that HB-EGF treatment restored whisker stimulation-induced functional hyperemia in CADASIL model mice, which is sensitive to Kir channel blocker, Ba²⁺. ** p<0.01, * p<0.05, NS; not significant by one-way ANOVA followed by Tukey's multiple comparisons test.

FIGS. 14A-14D show K⁺-evoked hyperemia is absent in CADASIL mice. FIG. 14A displays the positioning of a micropipette containing 10 mM K⁺ and TRITC-dextran (red) in close apposition to a capillary (green) in a Tg88 (CADASIL) mouse. K⁺ was locally ejected onto the capillary of interest during high frequency line scanning to measure RBC flux. FIG. 14B (top) shows raw recordings of RBC flux at baseline and after 10 mM K⁺ application to a capillary in a Tg129 (control) mouse, which increased flux. FIG. 14B (bottom) shows a full trace from the raw recordings shown in FIG. 14B. FIG. 14C shows, as in FIG. 14B, for a Tg88 (CADASIL) mouse. Here, K⁺ application had no effect on blood flow. FIG. 14D is the summary data indicating that K⁺ evoked hyperemia is crippled in Tg88 (CADASIL) mice (n=16-17 experiments in 7-8 mice; P=0.0014 (t31=3.504, unpaired Student's t-test).

FIGS. 15A-15F show the deficit of capillary-to-arteriole electrical signaling is restored by HB-EGF ex vivo. FIG. 15A show pipette positions (tip indicated by arrowheads) for arteriole stimulation (left) and capillary stimulation (right). FIG. 15B shows representative traces of arteriolar diameter in capillary-parenchymal arteriole (CaPA) preparations. Pressure ejection of 10 mM K⁺ (5 psi) onto capillaries (P2, purple) produced rapid upstream arteriolar dilation in the preparation from TgNotch3^{WT} (control) animal only, not in the preparation from TgNotch3^{R169C} (CADASIL) mouse. FIG. 15C shows the summary data indicating that K⁺ evoked upstream arteriolar dilation is present in TgNotch3^{WT} (control) animals (n=8 experiments in 8 mice) but crippled in TgNotch3^{R169C} (CADASIL) mice (n=8 experiments in 8 mice; unpaired Student's t-test). FIG. 15D shows a representative trace of arteriolar diameter in a capillary-parenchymal arteriole (CaPA) preparation from TgNotch3^{R169C} (CADASIL) mouse. Bath application of HB-EGF restored myogenic tone and upstream arteriolar diameter in response to capillary stimulation with 10 mM K. FIG. 15E shows the summary data in 5 different CaPA preparations. FIG. 15F shows the absence of effect of HB-EGF in a preparation from endothelial specific inward rectifier K⁺ (Kir) channel deficient mouse.

FIGS. 16A-16D show that Kir2.1 channel currents are suppressed in CADASIL cECs and can be corrected with HB-EGF. FIG. 16A shows representative traces of Kir2.1 current in freshly isolated mouse cECs bathed in 60 mM K⁺, recorded using voltage-ramps (−140 to 50 mV) using the perforated configuration. The upper tracing was recorded from a transgenic WT (TgNotch3^{WT}) cEC, and the bottom tracing was obtained from a CADASIL (TgNotch3^{R169C})cEC. FIG. 16B is summary data showing Kir2.1 currents at −140 mV in the perforated whole-cell configuration (intact cytoplasm) in TgNotch3^{WT} and TgNotch3^{R169C} cECs. Error bars represent SEM (n=11-24 cECs obtained from 3 or 4 mice). **P<0.01, unpaired Student's t test. FIG. 16C shows representative traces of Ba²⁺-subtracted Kir2.1 current in freshly isolated mouse CADASIL cECs bathed in 60 mM K⁺, recorded using voltage-ramps (−140 to 50 mV) using the perforated configuration. The upper tracing was recorded from a control CADASIL cEC, and the bottom from a CADASIL cEC incubated with HB-EGF (30 ng/ml) for 20 minutes.

FIG. 16D is summary data showing Kir2.1 currents at −140 mV in the perforated whole-cell configuration CADASIL cECs in the absence and presence of HB-EGF. Right bar graphs show no effect when TgNotch3^{WT} (TgWT) cECs were incubated with HB-EGF. Error bars represent SEM (n=6-12 cECs obtained from 5 mice). *P<0.05, unpaired Student's t test and ns denotes not significant.

FIGS. 17A-17G show excess of TIMP3 around brain capillary endothelial cells blunts Kir2.1-mediated electrical signaling through inhibition of the ADAM17/HB-EGF/EGFR module. FIG. 17A shows how pathogenic accumulation of TIMP3 blunts EGFR activation in CADASIL. FIG. 17B shows representative traces of arteriolar diameter in capillary-parenchymal arteriole (CaPA) preparations from TgNotch3^{WT} (control) mouse showing the progressive inhibition of the upstream arteriolar dilation in response to capillary stimulation with 10 mM K⁺ by batch application of recombinant TIMP3. FIG. 17C shows the summary data of 6 different CaPA preparations from 6 mice. FIG. 17D shows the restoration of capillary-to-arteriole electrical signaling in CaPA preparations by genetic reduction of TIMP3 expression and its inhibition by Kir channel blocker Ba²⁺. FIG. 17E shows summary data from 6 CaPA preparations from 6 different TgNotch3^{R169C}; Timp3^{+/-} mice and the complete inhibition of the dilation by Ba²⁺. FIG. 17F shows a representative trace of Ba²⁺-subtracted Kir2.1 current in freshly isolated mouse TgNotch3^{R169C}; Timp3^{+/-} cECs bathed in 60 mM K⁺, recorded using voltage-ramps (−140 to 40 mV) using the perforated configuration. FIG. 17G is summary data of inward Kir2.1 currents (at −140 mV) recorded from TgNotch3^{R169C} and TgNotch3^{R169C}; Timp3^{+/-} cECs (n=11-13 cECs obtained from 5 mice). ***P<0.001, unpaired Student's t test.

FIGS. 18A-18G show the restoration of capillary-to-arteriole electrical signaling by exogenous addition of soluble phosphatidylinositol 4,5-bisphosphate (PIP₂). FIG. 18A shows representative traces of Ba²⁺-subtracted Kir2.1 current recorded using the perforated configuration from a control TgNotch3^{R169C} cEC or a cEC pre-incubated with 10 μM diC16-PIP₂ for 20 minutes. FIG. 18B is summary data of inward Kir2.1 currents (at −140 mV) recorded from control TgNotch3^{R169C} and TgNotch3^{R169C} cECs treated with 10 μM diC16-PIP₂ (n=6-12 cECs obtained from 4 mice). **P<0.01, unpaired Student's t test. FIG. 18C shows representative traces and summary data of Kir2.1 current recorded using the perforated configuration from TgNotch3^{WT}, control TgNotch3^{R169C} or a TgNotch3^{R169C} cEC dialyzed with 10 μM diC8-PIP₂. FIG. 18C (right) is summary data of inward Kir2.1 currents (at −140 mV) recorded from control TgNotch3^{R169C} and TgNotch3^{R169C} cECs treated with 10 μM diC16-PIP₂ (n=9-13 cECs in each group). *P<0.05, **P<0.01, ***P<0.001 one-way ANOVA followed by Dunnett's multiple comparisons test. FIG. 18D (upper panel) shows PIP₂ labelled with fluorescent BODIPY group is integrated into capillary endothelial cell plasma membrane as illustrated by the remaining fluorescence after a 30 minutes wash. Fluorescence recovery after photobleaching (FRAP—lower panel) of a ˜10 μm² disk confirmed the mobility of PIP₂ in the plasma membrane. BODIPY-labelled PIP₂ displayed similar diffusion coefficient in preparations from TgNotch3^{WT} (n=11) and TgNotch3^{R169C}, (n=8) 2.63e-09 cm²/sec and 2.58e-09 cm²/sec, respectively. FIG. 18E shows a representative trace of arteriolar diameter in a capillary-parenchymal arteriole (CaPA) preparation from TgNotch3^{R169C} (CADASIL) mouse. Bath application of exogenous PIP₂ restored upstream arteriolar diameter in response to capillary stimulation with 10 mM K⁺. FIG. 18F shows the summary data in 4 different CaPA preparations. FIG. 18G shows the absence of effect of soluble PIP₂ in a preparation from endothelial specific inward rectifier K⁺ (Kir) channel deficient mouse, highlighting the necessary presence of Kir channels in capillary endothelial cells.

FIGS. 19A-19B show phosphatidylinositol 4,5-bisphosphate (PIP₂) enhanced whisker stimulation-induced functional hyperemia in CADASIL model mice. FIG. 19A shows representative traces of whisker stimulation-induced CBF change before and after PIP₂ treatment in CADASIL model (TgNotch3^{R169C}). FIG. 19B is the summary showing that whisker stimulation-induced functional hyperemia was increased after PIP₂ treatment.

FIGS. 20A-20B show that Kir2.1 channel activity in CADASIL is intact in arterial vascular cells. FIG. 20A shows representative traces of Kir2.1 current recorded before and after using the perforated configuration from a CADASIL or a TgWT arterial smooth muscle cells. FIG. 20A (right) shows representative traces of Kir2.1 current recorded in arterial ECs using 60 mM K⁺ in the bath solution. FIG. 20B is summary data of inward Kir2.1 currents (at −140 mV) recorded from arterial smooth muscle cells or endothelial cells obtained from TgWT or CADASIL mice. (n=8-12 cECs obtained from 7 mice). Unpaired Student's t test.

FIGS. 21A-21C show that exogenous PIP₂ has a negligible effect on isolated intracerebral arterioles diameter. FIGS. 21A and 21B show typical recordings of luminal diameter of pressurized parenchymal arterioles from TgNotch3^{WT} (control) and TgNotch3^{R169C} (CADASIL) mice. NS309 and U46619 are used to test the ability of the arteriole to dilate and constrict, respectively. Bath application of soluble PIP₂ at 10 μM has little effect on arteriole diameter. FIG. 21C shows the summary data from 6 TgNotch3^{WT} (control) mice and 5 TgNotch3R^{169C} (CADASIL) mice.

### DETAILED DESCRIPTION

The present application relates to method of treating a subject for a condition characterized by reduced cerebral blood flow. The method involves selecting a subject having a condition characterized by reduced cerebral blood flow and administering, to the selected subject, a therapeutic agent that increases the level of a phosphatidylinositol 4,5-bisphosphate (PIP₂), under conditions effective to treat the condition characterized by reduced cerebral blood flow.

In certain embodiments, the condition characterized by reduced cerebral blood flow is selected from small vessel disease, ischemic stroke, traumatic brain injury, and cerebral ischemia.

As described supra, ischemic conditions like stroke cause rapid neuronal cell death by severely reducing nutrient and oxygen supply. Immediately restoring blood flow following an ischemic event or a traumatic brain injury is therefore crucial for patient outcomes.

Similarly, “cerebral ischemia” or brain ischemia, refers to the reduction or cessation of blood flow to the central nervous system, which can be characterized as either global or focal. Global cerebral ischemia refers to reduction of blood flow within the cerebral vasculature resulting from systemic circulatory failure caused by, e.g., dementia, shock, cardiac failure, or cardiac arrest. Shock is the state in which failure of the circulatory system to maintain adequate cellular perfusion results in reduction of oxygen and nutrients to tissues. Within minutes of circulatory failure, tissues become ischemic, particularly in the heart and brain. Focal cerebral ischemia refers to cessation or reduction of blood flow within the cerebral vasculature resulting from a partial or complete occlusion in the intracranial or extracranial cerebral arteries. Such occlusion typically results in stroke, a syndrome characterized by the acute onset of a neurological deficit that persists for at least 24 hours, reflecting focal involvement of the central nervous system. Stroke is the result of a disturbance of the cerebral circulation. Other causes of focal cerebral ischemia include vasospasm due to subarachnoid hemorrhage or iatrogenic intervention.

As described supra, small vessel disease (SVD) of the brain is a leading cause of stroke and age-related cognitive decline and disability for which there are currently no treatments (Pantoni, “Cerebral Small Vessel Disease: From Pathogenesis and Clinical Characteristics to Therapeutic Challenges,” Lancet Neurology 9:689-701 (2010), which is hereby incorporated by reference in its entirety). Cerebral SVD refers to pathological processes that affect the structure or function of small vessels on the surface and within the brain, including arteries, arterioles, capillaries, venules and veins. The consequences of pathological changes of small vessels of the brain include white matter hyperintensities, small infarctions or hemorrhages in white and/or deep gray matter, enlargement of perivascular spaces, and brain atrophy (Joutel et al., “Cerebral Small Vessel Disease: Insights and Opportunities From Mouse Models of Collagen IV-Related Small Vessel Disease and Cerebral Autosomal Dominant Arteriopathy with Subcortical Infarcts and Leukoencephalopathy,” Stroke 45:1215-1221 (2014), which is hereby incorporated by reference in its entirety). Cerebral autosomal-dominant arteriopathy with subcortical infarcts and leukoencephalopathy (CADASIL) is the most common hereditary cerebral SVD.

Accordingly, the present application also relates to a method of treating cerebral autosomal-dominant arteriopathy with subcortical infarcts and leukoencephalopathy (CADASIL) in a subject. The method involves selecting a subject having cerebral autosomal-dominant arteriopathy with subcortical infarcts and leukoencephalopathy (CADASIL) and administering, to the selected subject, a therapeutic agent that increases the level of a phosphatidylinositol 4,5-bisphosphate (PIP₂), under conditions effective to treat CADASIL in the selected subject.

CADASIL (for cerebral autosomal dominant arteriopathy with subcortical infarcts and leukoencephalopathy; or: CADASIL syndrome) causes a type of lacunar syndrome accompanied by obliviousness whose key features include recurrent sub-cortical ischemic events and vascular dementia and which is associated with diffuse white-matter abnormalities on neuro-imaging. CADASIL is inherited in an autosomal dominant manner.

As used herein, the term “treat” refers to the application or administration of the therapeutic agent of the present application to a subject, e.g., a patient. The treatment can be to cure, heal, alleviate, relieve, alter, remedy, ameliorate, palliate, improve or affect the cerebral blood flow, or the symptoms of the condition characterized by reduced cerebral blood flow (i.e., conditions such as, but not limited to, small vessel disease, ischemic stroke, traumatic brain injury, and cerebral ischemia).

As used herein, the term “subject” is intended to include human and non-human animals. Non-human animals include all vertebrates, e.g., mammals and non-mammals, such as non-human primates, sheep, dog, cow, chickens, amphibians, reptiles, etc.

As used herein, “increases the level of phosphatidylinositol 4,5-bisphosphate” refers to an increase in membrane PIP₂ by at least about 5%, 10%, 15%, 20%, 25%, 30%, 35%, 40%, 45%, 50%, 55%, 60%, 65%, 70%, 75%, 80%, 85%, 90%, or 95%.

In certain embodiments, the level of PIP₂ is increased within the membrane of capillary endothelial cells.

Capillary endothelial cells are sensors of neural activity that integrate sensory information to translate it into changes in cerebral blood flow. In particular, capillary endothelial cells contain inward rectifier K⁺ (Kir) channels, which are involved in driving vasorelaxation and a local increase in cerebral blood flow when activated by increased K⁺. This is known as functional hyperemia. Functional hyperemia is sustained by local increases in cerebral blood flow that accompanies neuronal activity to satisfy enhanced glucose and oxygen demands. This is also known as neurovascular coupling (NVC).

Accordingly, the present application also relates to methods of restoring cerebral blood flow and functional hyperemia in a subject. These methods involve selecting a subject having reduced cerebral blood flow or reduced functional hyperemia and administering, to the selected subject, a therapeutic agent that increases the level of PIP₂, under conditions effective to restore cerebral blood flow or functional hyperemia.

Subjects having reduced cerebral blood flow and/or reduced functional hyperemia include, without limitation, subjects having small vessel disease, ischemic stroke, traumatic brain injury, and cerebral ischemia. Other conditions associated with reduced functional hyperemia include hypertension, hypotension, autonomic dysfunction, spinal cord injury, Alzheimer's disease, smoking, diabetes, and healthy aging.

In the methods of the present application, the levels of cerebral blood flood and/or functional hyperemia are restored to about 5%, 10%, 15%, 20%, 25%, 30%, 35%, 40%, 45%, 50%, 55%, 60%, 65%, 70%, 75%, 80%, 85%, 90%, 95%, or 100% of the levels present in a healthy subject.

Methods for measuring cerebral blood flow are known in the art. Three non-portable methods that are presently used include: 1) injecting radioactive xenon into the cervical carotid arteries and observing the radiation it emits as it spreads throughout the brain; 2) positron emission tomography, also based on the injection of radioactive material; and 3) magnetic resonance angiography. A fourth method, transcranial Doppler (TCD) uses ultrasound and is not invasive, and gives immediate results.

Functional hyperemia (attributable to neurovascular coupling) can be measured using methods known in the art including, but not limited to, transcranial Doppler (TCD) and near infrared spectroscopy (NIRS). Such methods are described in Phillips et al., “Neurovascular Coupling in Humans: Physiology, Methodological Advances and Clinical Implications,” Journal of Cerebral Blood Flow and Metabolism 36(4):647-664 (2016), which is hereby incorporated by reference in its entirety.

The methods of the present application include administering, to a subject, a therapeutic agent that increases the level of a phosphatidylinositol 4,5-bisphosphate (PIP₂). PIP₂ is a lipid in the family of phosphoinositides. Phosphoinositides (“PIs”) are a family of minority acidic phospholipids in cell membranes and serve as signaling molecules in a diverse array of cellular pathways. Aberrant regulation of PIs in certain cell types has been shown to promote various human disease states (Pendaries et al., “Phosphoinositide Signaling Disorders in Human Diseases,” FEBS Lett. 546(1):25-31 (2003), which is hereby incorporated by reference in its entirety). PI signaling is mediated by the interaction with signaling proteins harboring the many specialized PI-binding domains. The interaction between these PI-binding domains and their target PIs results in the recruitment of the lipid-protein complex into the intracellular membrane.

PI signaling is tightly regulated by a number of kinases, phosphatases, and phospholipases. In the central nervous system, the levels of PIs in nerve terminals are regulated by specific synaptic kinases, such as phosphoinositol phosphate kinase type 1γ (PIPk1γ) and phosphatases, such as synaptojanin 1 (SYNJ1). PIP₂ hydrolysis in the brain occurs in response to stimulation of a large number or receptors via two major signaling pathways: a) the activation of G-protein linked neurotransmitter receptors (e.g. glutamate and acetylcholine), mediated by phospholipase C (PLC), and b) the activation of tyrosine kinase linked receptors for growth factors and neurotrophins (e.g. NGF, BDNF), mediated by PLC. The reaction produces two intracellular messengers, IP3 and diacylglycerol (DAG), which mediate intracellular calcium release and protein kinase C (PKC) activation, respectively. Moreover, and as described herein, localized membrane changes in PIP₂ itself is an important signal as PIP₂ is a modulator of a variety of channels and transporters (Hilgemann et al., “The Complex and Intriguing Lives of PIP₂ with Ion Channels and Transporters,” STKE 111:1-8 (2001), which is hereby incorporated by reference in its entirety).

In one embodiment, the therapeutic agent that increases the level of PIP₂ is a small molecule.

As used herein, “small molecules” are typically organic, non-peptide molecules, having a molecular weight less than 10,000 Da, preferably less than 5,000 Da, more preferably less than 1,000 Da, and most preferably less than 500 Da. This class of modulators includes chemically synthesized molecules, for instance, compounds from combinatorial chemical libraries.

As described supra, regulation of PIP₂ in the brain is controlled by the activity of G-protein coupled receptors and activation of tyrosine kinase linked receptors, both of which involve stimulation of PLC. Accordingly, small molecules which inhibit GqPCR and/or tyrosine kinase linked receptors and/or PLC, thereby inhibiting hydrolysis of PIP₂, are contemplated for use in the methods of the present application.

Inhibitors of PLC are known in the art and include, without limitation, edelfosine, or a derivative thereof; miltefosine, or a derivative thereof; a phospholipid derivative as described in German Patent DE 4222910, which is hereby incorporated by reference in its entirety, such as, but not limited to, perifosine; ilmofosine, or a derivative thereof; BN 52205 (Principe et al., “Tumor Cell Kinetics Following Long-Term Treatment with Antineoplastic Ether Phospholipids,” Cancer Detection and Prevention 18(5):393-400 (1994), which is hereby incorporated by reference in its entirety), or a derivative thereof; BN 5221.1 (Principe et al., “Tumor Cell Kinetics Following Long-Term Treatment with Antineoplastic Ether Phospholipids,” Cancer Detection and Prevention 18(5):393-400 (1994), which is hereby incorporated by reference in its entirety), or a derivative thereof; and 2-fluoro-3-hexadecyloxy-2-methylprop-1-yl 2′-(trimethylammonio) ethyl phosphate (Haufe et al., “Synthesis of a Fluorinated Ether Lipid Analagous to a Platelet Activating Factor,” Eur. J. Organic Chem. 23:4501-4507 (2001), which is hereby incorporated by reference in its entirety) or a derivative thereof

Other exemplary small molecules useful as therapeutic agents that increase the level of PIP₂ include, without limitation, an erucyl, brassidyl, or nervonyl-containing phosphocholine as described in European Patent No. 507337, which is hereby incorporated by reference in its entirety, such as, but not limited to, erucylphosphocholine, or a derivative thereof; an alkylphosphocholine, including, but not limited to, the alkylphosphocholines disclosed in U.S. Pat. No. 4,837,023, which is hereby incorporated by reference in its entirety, e.g. hexadecylphosphocholine, or a derivative thereof; and LY294002 (Schmid et al., “Phosphatases as Small Molecule Target: Inhibiting the Endogenous Inhibitors of Kinases,” Biochem. Soc. Trans. 32(part 2):348-349 (2004), which is hereby incorporated by reference in its entirety; Shingu et al., “Growth Inhibition of Human Malignant Glioma Cells Induced by the PI3-K-Specific Inhibitor,” J. Neurosurg. 98(1):154-161 (2003), which is hereby incorporated by reference in its entirety).

In another embodiment, the therapeutic agent that increases the level of PIP₂ is a soluble PIP₂ analog.

Soluble PIP₂ analogs have been described in the art (see, e.g., U.S. Patent Application Publication No. 2005/0148042 to Prestwich et al.; Bru et al., “Development of a Solid Phase Synthesis Strategy for Soluble Phosphoinositide Analogues,” Chemical Science 6 (2012); Chen et al., “Asymmetric Synthesis of Water-Soluble, Nonhydrolyzable Phosphonate Analogue of Phosphatidylinositol 4,5-Bisphosphate,” Journal of Organic Chemistry 63(3):430-431 (1998), which are hereby incorporated by reference in their entirety).

Exemplary soluble PIP₂ analogs for use in the methods of the present application include, without limitation, diC4-PIP₂, diC6-PIP₂, diC8-PIP₂ (08:0 PIP₂), diC16-PIP₂, diC18:1 PIP₂, 18:0-20:4 PIP₂, and brain PIP₂.

Other methods for increasing the levels of PIP₂ are contemplated as well. As described in Capone et al., “Mechanistic Insights into a TIMP3-Sensitive Pathway Constitutively Engaged in the Regulation of Cerebral Hemodynamics,” eLife 5:e17536 (2016), which is hereby incorporated by reference in its entirety, the ADAM17/HB-EGF/EGFR/Kv signaling pathway also plays a central role in the physiological and pathological control of cerebral blood flow and arterial tone. Members of this pathway are regulated by the protein TIMP3, which has been shown to be involved in CADASIL (Monet-Leprêtre et al., “Abnormal Recruitment of Extracellular Matrix Proteins by Excess Notch3 ECD: a New Pathomechanism in CADASIL,” Brain 136:1830-1845 (2013), which is hereby incorporated by reference in its entirety). Accordingly, in view of the Examples infra, therapeutic agents which modulate proteins involved in the ADAM17/HB-EGF/EGFR/Kv signaling pathway are also contemplated for use in the methods of the present application. By way of example, HB-EGF may be administered to affect PIP₂ levels.

It will be appreciated that the exact dosage of the therapeutic agent of the present application is chosen by the individual physician in view of the patient to be treated. In general, dosage and administration are adjusted to provide an effective amount of the agent to the patient being treated. As used herein, the “effective amount” of a therapeutic agent refers to the amount necessary to elicit the desired biological response. As will be appreciated by those of ordinary skill in this art, the effective amount of therapeutic agent of the present application may vary depending on such factors as the desired biological endpoint, the drug to be delivered, the target tissue, the route of administration, etc. Additional factors which may be taken into account include the severity of the disease state; age, weight and gender of the patient being treated; diet, time and frequency of administration; drug combinations; reaction sensitivities; and tolerance/response to therapy.

An “effective amount” may also be a “a prophylactically effective amount,” which refers to an amount of the therapeutic agent as described herein, which is effective, upon single- or multiple-dose administration to the subject, in preventing or delaying the occurrence of the onset or recurrence of a disorder, e.g., reduced cerebral blood flow, or treating a symptom thereof.

Dosages for administration of exemplary therapeutic agents include, but are not limited to, (i) edelfosine, or a derivative thereof, e.g., at a daily dose of between about 1-25 mg/kg/day and preferably between about 5-20 mg/kg/day, or in an amount to produce a local concentration of between 1 and 50 μM and preferably between 5 and 20 μM; (ii) miltefosine, or a derivative thereof, e.g., at a dose of about 2.5 mg/kg/day, and/or a 10 mg or 50 mg tablet administered orally once or twice a day; (iii) a phopholipid derivative such as, but not limited to, perifosine; (iv) an erucyl, brassidyl or nervonyl-containing phosphocholine such as, but not limited to, erucylphosphocholine, or a derivative thereof, e.g., at a daily dose of about 0.5-10 millimoles; (v) an alkylphosphocholine, including, but not limited to, the alkylphosphocholines e.g. hexadecylphosphocholine, e.g., at a dose of about 5 to 2000 mg, and preferably between about 5 and 100 mg, per day; (vi) ilnofosine, or a derivative thereof, e.g., at a dose of 12-650 mg/m²/week or 10/mg/kg per day; (vii) BN 52205 or a derivative thereof; (viii) BN 5221.1 or a derivative thereof, (ix) 2-fluoro-3-hexadecyloxy-2-methylprop-1-yl 2′-(trimethylammonio) ethyl phosphate or a derivative thereof, and (x) LY294002 or a derivative thereof, e.g., at a dose that provides a local concentration of 2-40 The foregoing dosages are provided as examples and do not limit the invention as regards effective doses of the recited compounds.

In practicing the methods of the present application, the administering step is carried out to treat a condition (i.e., a condition characterized by reduced cerebral blood flow and CADASIL) or effect a physiological change (i.e., restore cerebral blood flow or functional hyperemia) in a subject. Such administration can be carried out systemically or via direct or local administration to the brain. By way of example, suitable modes of systemic administration include, without limitation orally, topically, transdermally, parenterally, intradermally, intracisternally, intramuscularly, intraperitoneally, intravenously, subcutaneously, or by intranasal instillation, by intracavitary or intravesical instillation, intraocularly, intraarterially, intralesionally, or by application to mucous membranes. Suitable modes of local administration include, without limitation, catheterization, implantation, direct injection, dermal/transdermal application, or portal vein administration to relevant tissues, or by any other local administration technique, method or procedure generally known in the art. The mode of affecting delivery of the therapeutic agent will vary depending on the type of the therapeutic agent (e.g., a small molecule) and the disease to be treated.

The therapeutic agent of the present application may be orally administered, for example, with an inert diluent, or with an assimilable edible carrier, or it may be enclosed in hard or soft shell capsules, or it may be compressed into tablets, or they may be incorporated directly with the food of the diet. The therapeutic agent of the present application may also be administered in a time release manner incorporated within such devices as time-release capsules or nanotubes. Such devices afford flexibility relative to time and dosage. For oral therapeutic administration, the agents of the present application may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of the therapeutic agent of the present application in such therapeutically useful compositions is such that a suitable dosage will be obtained.

When the therapeutic agent of the present application is administered parenterally, solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the therapeutic agent of the present application systemically, it may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the therapeutic of the present application can also be achieved using infusion pump devices. Such devices allow continuous infusion of desired compounds avoiding multiple injections and multiple manipulations.

In addition to the formulations described previously, the therapeutic agent may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

### Examples

The examples below are intended to exemplify the practice of embodiments of the disclosure but are by no means intended to limit the scope thereof.

### Materials and Methods for Examples 1-5

Animals. Adult (2- to 3-mo-old) male C57BL/6J mice (The Jackson Laboratory) were group-housed on a 12-h light:dark cycle with environmental enrichment and free access to food and water. All animals were euthanized by i.p. injection of sodium pentobarbital (100 mg/kg), followed by rapid decapitation. All procedures received prior approval from the University of Vermont Institutional Animal Care and Use Committee.

Chemicals. 5-[(Cyclohexylcarbonyl)amino]-2-(phenylamino)-thiazolecarboxamide (UNC-3230), and N,N,N-trimethyl-4-(2-oxo-1-pyrolidinyl)-2-butyn-1-ammonium iodide (oxotremorine M) were obtained from Tocris Bioscience. 1,2-Dioctanoyl phosphatidylinositol 4,5-bisphosphate sodium salt (diC8-PIP₂) was purchased from Cayman Chemical, and 12-(2-cyanoethyl)-6,7,12,13-tetrahydro-13-methyl-5-oxo-5H-indolo(2,3-a)pyrrolo(3,4-c)-carbazole (Gö6976) was from Calbiochem. Unless otherwise noted, all other chemicals were obtained from Sigma-Aldrich.

Capillary Endothelial Cell Isolation. Single capillary endothelial cells (cECs) were obtained from mouse brains by mechanical disruption of two 160-μm-thick brain slices using a Dounce homogenizer, as previously described (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety). Slices were homogenized in ice-cold artificial cerebrospinal fluid, with the composition 124 mM NaCl, 3 mM KCl, 2 mM CaCl₂, 2 mM MgCl₂, 1.25 mM NaH₂PO₄, 26 mM NaHCO₃, and 4 mM glucose. Debris was removed by passing the homogenate through a 62-μm nylon mesh. Retained capillary fragments were washed into dissociation solution, composed of 55 mM NaCl, 80 mM Na-glutamate, 5.6 mM KCl, 2 mM MgCl₂, 4 mM glucose, and 10 mM Hepes (pH 7.3) containing neutral protease (0.5 mg/mL), elastase (0.5 mg/mL; Worthington), and 100 μM CaCl₂, and incubated for 24 min at 37° C. Following this step, 0.5 mg/mL collagenase type I (Worthington) was added, and the solution was incubated for an additional 2 min at 37° C. The suspension was filtered and washed to remove enzymes, and single cells and small capillary fragments were dispersed by triturating four to seven times with a fire-polished glass Pasteur pipette. Cells were used within ˜6 h after dispersion.

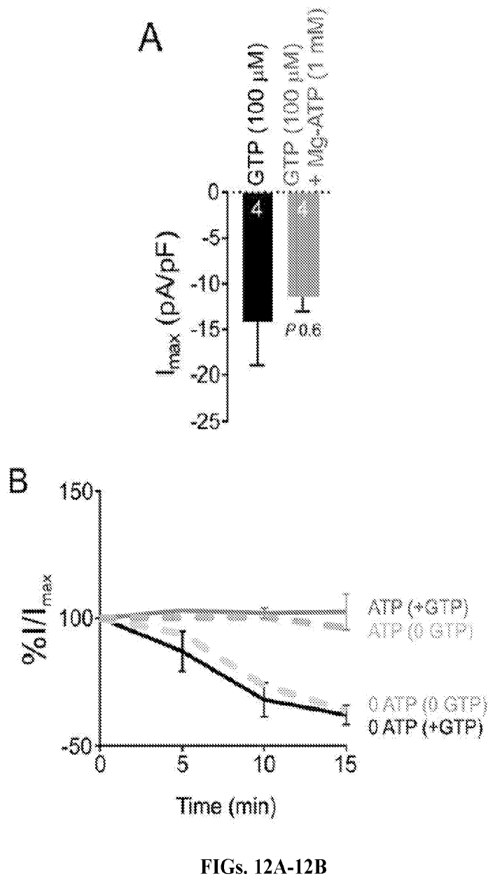

Electrophysiology. Whole-cell currents were recorded using a patch-clamp amplifier (Axopatch 200B; Molecular Devices), filtered at 1 kHz, digitized at 5 kHz, and stored on a computer for offline analysis with Clampfit 10.3 software. Whole-cell capacitance was measured using the cancellation circuitry in the voltage-clamp amplifier. Electrophysiological analyses were performed in either the conventional or perforated whole-cell configuration. Recording pipettes were fabricated by pulling borosilicate glass (1.5-mm outer diameter, 1.17-mm inner diameter; Sutter Instruments) using a Narishige puller. Pipettes were fire-polished to a tip resistance of ˜4 to 6 MΩ. The bath solution consisted of 80 mM NaCl, 60 mM KCl, 1 mM MgCl₂, 10 mM HEPES, 4 mM glucose, and 2 mM CaCl₂ (pH 7.4). For the conventional whole-cell configuration, pipettes were backfilled with a solution consisting of 10 mM NaOH, 11.4 mM KOH, 128.6 mM KCl, 1.1 mM MgCl₂, 2.2 mM CaCl₂, 5 mM EGTA, and 10 mM HEPES (pH 7.2). As noted in the Examples below, the pipette solution was supplemented in some experiments with ATP (10 μM, 100 μM, or 1 mM) or ATP-γ-S(1 mM). In a subset of experiments (FIG. 12), Na-GTP (100 μM) was added to the pipette solution alone or together with 1 mM Mg-ATP; in neither setting did Na-GTP have an effect on peak Kir2.1 current amplitude or the kinetics of current decline. In a subset of experiments, BAPTA (5.4 mM) was used in place of EGTA. For perforated-patch electro-physiology, the pipette solution was composed of 10 mM NaCl, 26.6 mM KCl, 110 mM K+ aspartate, 1 mM MgCl₂, 10 mM HEPES, and 200 to 250 μg/mL amphotericin B, added freshly on the day of the experiment.

Ex Vivo Capillary-Parenchymal Arteriole Preparation. The capillary-parenchymal arteriole (CaPA) preparation was obtained by dissecting parenchymal arterioles arising from the M1 region of the middle cerebral artery, leaving the attached capillary bed intact, as reported recently (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety). Precapillary arteriolar segments were cannulated on glass micropipettes on a Living Systems Instrumentation pressure myograph, with one end occluded by a tie. The ends of the capillaries were then sealed by the downward pressure of an overlying glass micropipette. Application of pressure (40 mmHg) to the cannulated parenchymal arteriole segment in this preparation pressurized the entire tree and induced myogenic tone in the parenchymal arteriole segment. With this preparation, 10 mM K⁺ was applied onto capillaries by pressure ejection from a glass micropipette (tip diameter, ˜5 μm) attached to a Picospritzer III (Parker) at ˜5 psi for 18 s. Luminal diameter in parenchymal arterioles was acquired in one region of the arteriolar segment at 15 Hz using IonWizard 6.2 edge-detection software (IonOptix). Changes in arteriolar diameter were calculated from the average luminal diameter measured over the last 10 s of stimulation and were normalized to the maximum dilatory responses in 0 mM Ca²⁺ bath solution at the end of each experiment.

In Vivo Cerebrovascular and Hemodynamics Imaging. Mice were anesthetized with isoflurane (5% induction, 2% maintenance), essentially as described previously (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety). Upon obtaining surgical-plane anesthesia, the skull was exposed, and a stainless-steel head plate was attached over the left hemisphere using dental cement. The head plate was secured in a holding frame, and a small (˜2-mm diameter) circular cranial window was drilled in the skull above the somatosensory cortex. Approximately 150 μL of a 3-mg/mL solution of FITC-dextran (molecular mass, 2,000 kDa) in saline was systemically administered via intravascular injection into the retroorbital sinus to enable visualization of the cerebral vasculature and contrast imaging of RBCs. Upon conclusion of surgery, isoflurane anesthesia was replaced with α-chloralose (50 mg/kg) and urethane (750 mg/kg). Body temperature was maintained at 37° C. throughout the experiment using an electric heating pad. Penetrating arterioles were first identified by observing RBCs flowing into the brain (as opposed to out of the brain via venules), and capillaries downstream of arterioles were selected for study. A pipette was next introduced into the solution covering the exposed cortex, and the duration and pressure of ejection were calibrated (300 ms, ˜8 to 10 psi) to obtain a small solution plume (radius, ˜10 μm). The pipette was maneuvered into the cortex and positioned adjacent to the capillary under study (mean depth, ˜73 μm), after which agents were ejected directly onto the capillary. Placement of the pipette in the brain as described restricted agent delivery to the capillary under study and caused minimal displacement of the surrounding tissue. Spatial coverage of the ejected solution was monitored by including 1.6 mg/mL tetramethylrhodamine isothiocyanate (TRITC; 150 kDa)-labeled dextran. RBC flux data were collected by line-scanning the capillary of interest at 5 kHz. Images were acquired using a Zeiss LSM-7 multiphoton microscope (Zeiss) equipped with a Zeiss 20× Plan Apochromat 1.0 N.A. DIC VIS-IR water-immersion objective and coupled to a Coherent Chameleon Vision II Titanium-Sapphire pulsed infrared laser (Coherent). FITC and TRITC were excited at 820 nm, and emitted fluorescence was separated through 500- to 550-nm and 570- to 610-nm bandpass filters, respectively.

Data Analysis. Data are expressed as means±SEM. Where appropriate, paired or unpaired t tests or analysis of variance (ANOVA) was performed using Graphpad Prism 7.01 software to compare the effects of a given condition or treatment. P values of <0.05 were considered statistically significant. Patch-clamp data were additionally analyzed using Clampfit 10.5 software.

### Example 1—Kir2.1 Channel Activity in Capillary Endothelial Cells is Sustained by an ATP-Dependent Mechanism

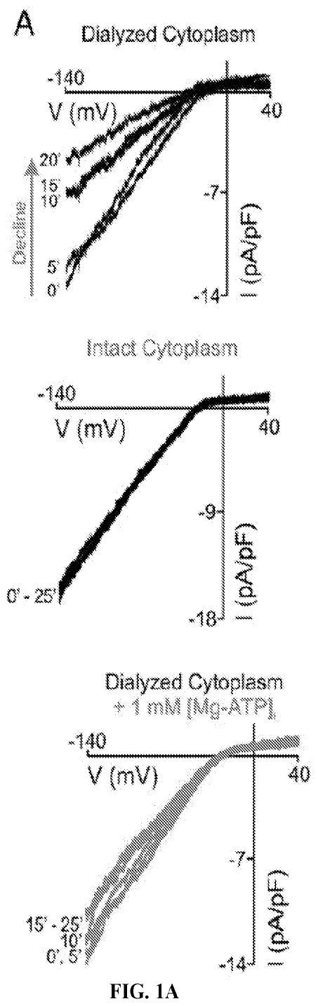

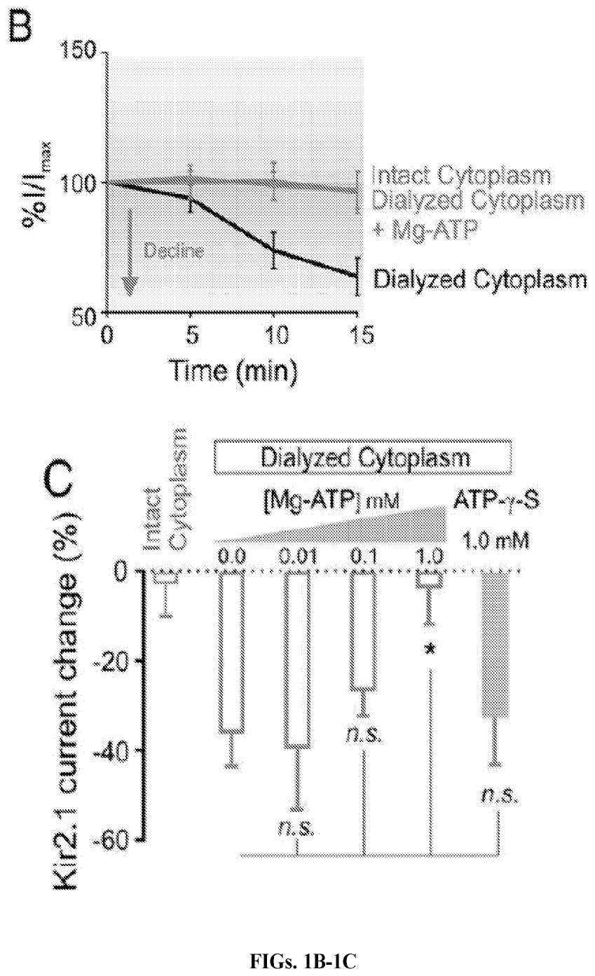

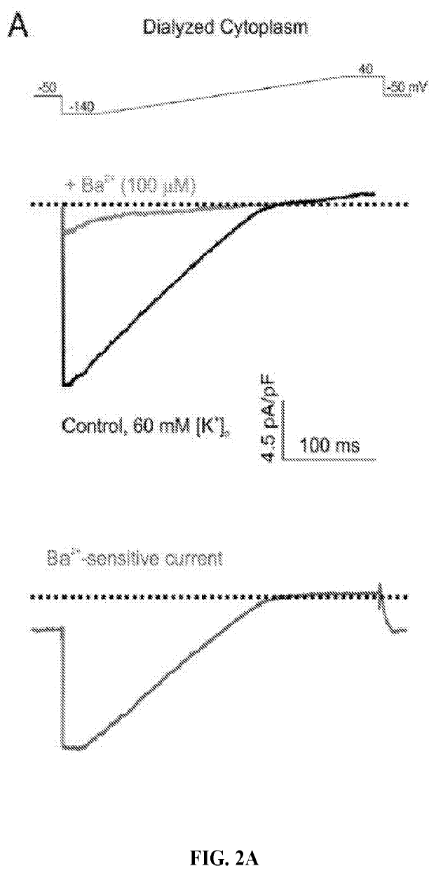

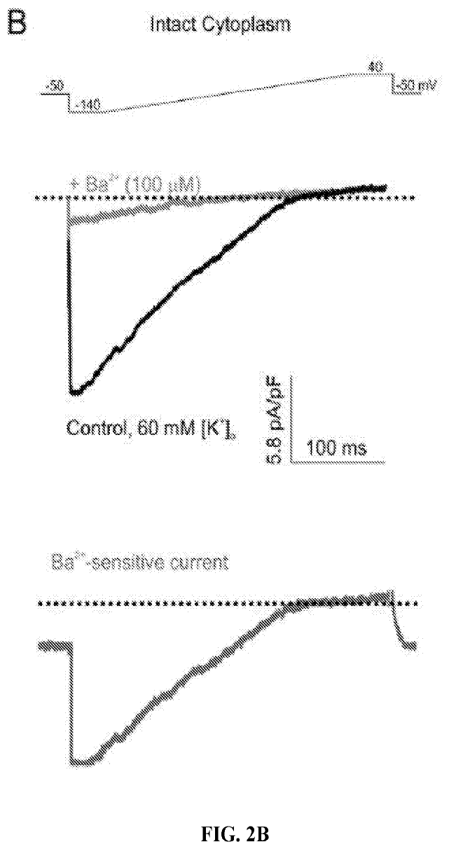

Recent work has demonstrated that Kir2.1 channels in capillary endothelial cells transduce electrical (hyperpolarizing) signals that rapidly dilate upstream arterioles and increase RBC flux, effects that are abrogated by selective knockdown of endothelial Kir2.1 channels (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety). Here, intracellular regulatory features of this Kir2.1 channel-dependent signaling mechanism was investigated. Kir2.1 currents were measured in freshly isolated C57BL/6J mouse brain capillary endothelial cells bathed in a 60-mM [K⁺]ₒ solution, used to increase Kir2.1 current amplitude. Under these conditions, the K⁺ equilibrium potential (E_{K}) was |23 mV. Ionic currents were recorded in the voltage-clamp mode of the patch-clamp technique. A 300-ms voltage-ramp protocol (−140 to +40 mV from a holding potential of −50 mV) was applied, and currents were recorded using the conventional whole-cell configuration. Inward K⁺currents were detected at potentials negative to EK with little outward current positive to EK, a characteristic feature of Kir2.1 channels (FIG. 1A). Intriguingly, Kir2.1 currents gradually declined after electrical access to the cell interior was attained. Because the conventional whole-cell configuration allows exchange of intracellular contents with the patch pipette solution, this observation suggested that a factor necessary for the maintenance of Kir2.1 channel activity was dialyzed out of the cell. In support of this interpretation, Kir2.1 currents were sustained in experiments performed using the perforated-patch configuration, in which the cytoplasm remains intact (FIG. 1A). Under both conditions, these currents were abolished by the Kir channel blocker Ba²⁺ (100 μM) (FIGS. 2A-2B), consistent with previous reports (Longden et al, “Capillary K⁺-Sensing Initiates Retrograde Hyperpolarization to Increase Local Cerebral Blood Flow,” Nat. Neurosci. 20:717-726 (2017); Quayle et al., “Inward Rectifier K⁺ Currents in Smooth Muscle Cells from Rat Resistance-Sized Cerebral Arteries,” Am. J. Physiol. 265:C1363-C1370 (1993); Hibino H, et al., “Inwardly Rectifying Potassium Channels: Their Structure, Function, and Physiological Roles,” Physiol. Rev. 90:291-366 (2010); Zaritsky et al., “Targeted Disruption of Kir2.1 and Kir2.2 Genes Reveals the Essential Role of the Inwardly Rectifying K⁺ Current in K⁺-Mediated Vasodilation,” Circ. Res. 87:160-166 (2000), which is hereby incorporated by reference in its entirety).

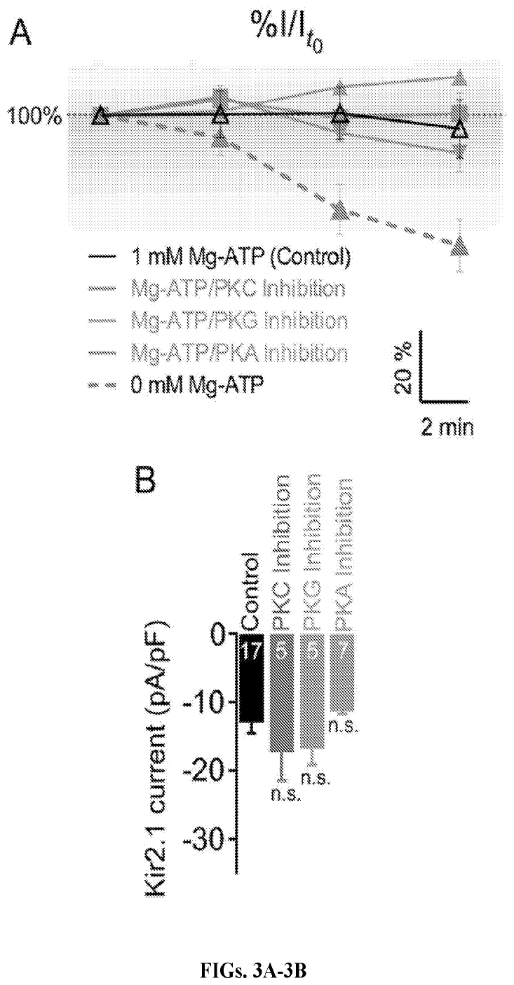

The pipette solution used for initial whole-cell patch-clamp experiments lacked ATP, a fortuitous omission that led us to focus on a potential ATP-dependent mechanism in regulating Kir2.1 channel activity. Under these original conditions, Kir2.1 currents measured in cells dialyzed with a solution lacking Mg-ATP declined by ˜36% after 15 min compared with those recorded immediately after acquisition of whole-cell electrical access (time=t₀). In contrast, Kir2.1 currents recorded with 1 mM Mg-ATP included in the pipette (intracellular) solution showed no decrease over the same time frame (FIG. 1A-1B). The decline in Kir2.1 currents was sensitive to the intracellular concentration of ATP, such that lower levels of Mg-ATP (10 or 100 μM) in the patch pipette were insufficient to prevent it (FIG. 1C). In addition, Mg-ATP-γ-S(1 mM), a nonhydrolyzable analog of ATP, failed to avert current decay (FIG. 1C), implying that ATP hydrolysis is required to sustain Kir2.1 currents and suggesting the involvement of a kinase. However, pharmacological inhibitors of protein kinase C (PKC), G (PKG), or A (PKA) in the presence of 1 mM Mg-ATP (intracellular), which is substantially higher than the KM, ATP (Michaelis constant for ATP) for these protein kinases (Knight et al., “Features of Selective Kinase Inhibitors,” Chem. Biol. 12: 621-637 (2005), which is hereby incorporated by reference in its entirety), had no significant effect on Kir2.1 current decline (FIGS. 3A-3B), arguing against a role for these protein kinases in sustaining capillary Kir2.1 activity.

### Example 2—Maintenance of PIP₂ Levels Through ATP-Dependent Phosphatidylinositol Kinase Activity Underlies Sustained Kir2.1 Channel Activity

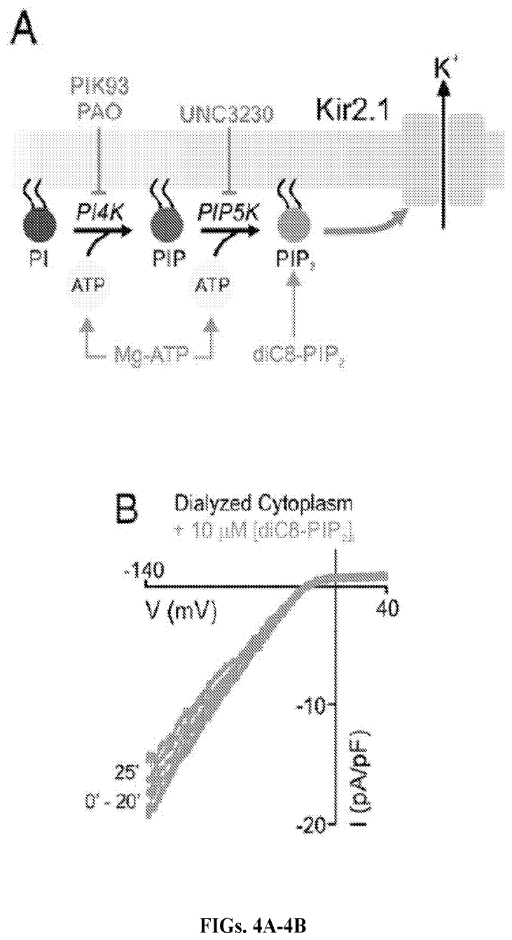

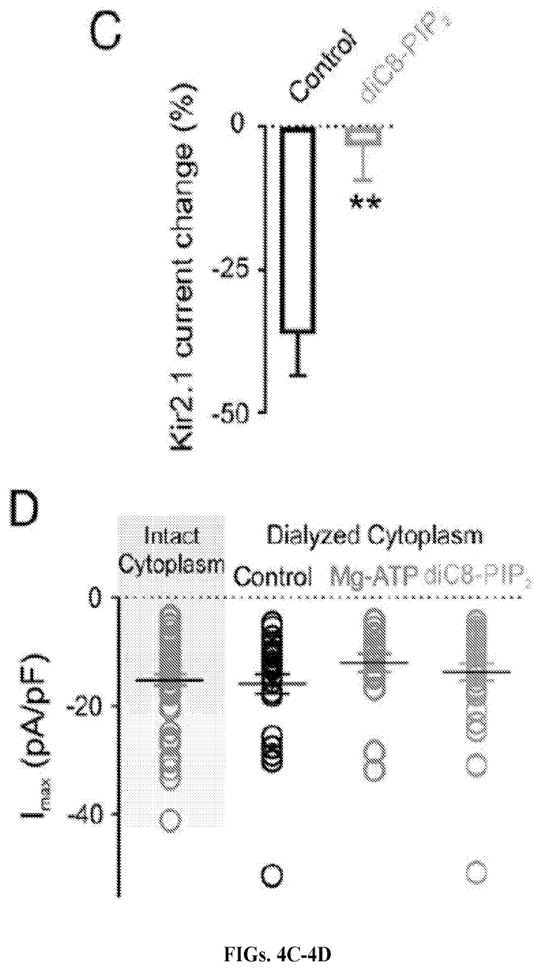

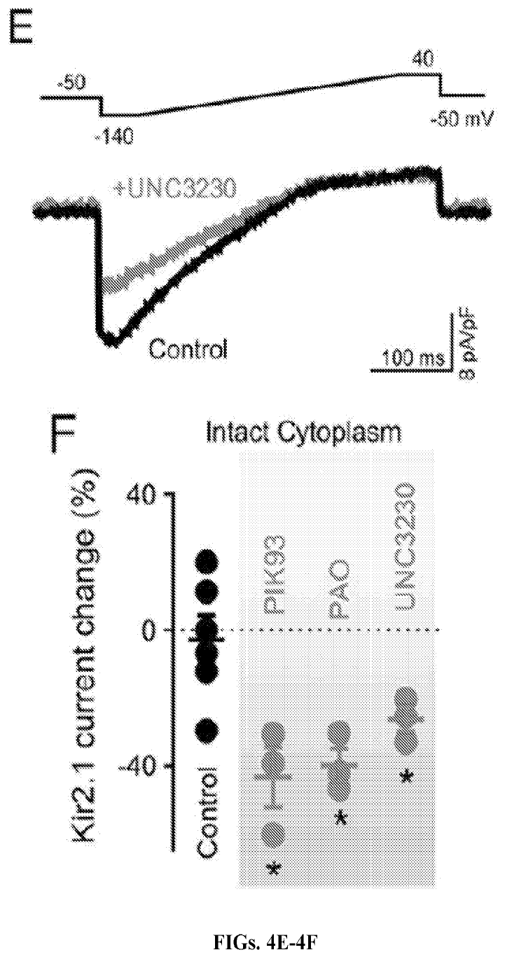

Unlike protein kinases, most of which are maximally activated by low micromolar ATP concentrations, lipid kinases generally require much higher concentrations of ATP to support their activity (Knight et al., “Features of Selective Kinase Inhibitors,” Chem. Biol. 12: 621-637 (2005); Hilgemann D W “Cytoplasmic ATP-Dependent Regulation of Ion Transporters and Channels: Mechanisms and Messengers,” Annu. Rev. Physiol. 59:193-220 (1997); Suer et al., “Human Phosphatidylinositol 4-Kinase Isoform PI4K92. Expression of the Recombinant Enzyme and Determination of Multiple Phosphorylation Sites,” Eur. J. Biochem. 268:2099-2106 (2001); Balla et al., “Phosphatidylinositol 4-Kinases: Old Enzymes with Emerging Functions,” Trends Cell Biol. 16:351-361 (2006), which are hereby incorporated by reference in their entirety). In light of the concentration dependence of intracellular ATP effects, noted above (FIG. 1C), and the well-known role of the phosphoinositide PIP₂ in regulating membrane proteins, including ion channels, attention was turned to the phosphoinositide pathway. Endogenous PIP₂ levels are dynamically regulated by the opposing actions of lipid kinases and phosphatases (Hille et al., “Phosphoinositides Regulate Ion Channels,” Biochim Biophys Acta 1851:844-856 (2015); Hilgemann D W “Cytoplasmic ATP-Dependent Regulation of Ion Transporters and Channels: Mechanisms and Messengers,” Annu Rev Physiol 59:193-220 (1997), which are hereby incorporated by reference in their entirety). The formation of PIP₂ reflects the sequential actions of phosphatidylinositol 4-kinase (PI4K), which converts phosphatidylinositol (PI) to phosphatidylinositol 4-phosphate (PIP), and phosphatidylinositol 4-phosphate 5-kinase (PIP5K), which converts PIP to PIP₂ (FIG. 4A). Phosphorylation of PI by PI4K is the rate-limiting step in PIP₂ synthesis, and Mg-ATP is required for the activity of PI4K (KM, ATP 0.4 to 1 mM) (Suer et al., “Human Phosphatidylinositol 4-Kinase Isoform PI4K92. Expression of the Recombinant Enzyme and Determination of Multiple Phosphorylation Sites,” Eur J Biochem 268:2099-2106 (2001); Balla et al., “Phosphatidylinositol 4-Kinases: Old Enzymes with Emerging Functions,” Trends Cell Biol 16:351-361 (2006); Gehrmann T, et al., “Functional Expression and Characterisation of a New Human Phosphatidylinositol 4-Kinase PI4K230,” Biochim Biophys Acta 1437:341-356 (1999), which are hereby incorporated by reference in their entirety). To determine whether the decline in Kir2.1 channel activity observed in the absence of Mg-ATP could be traced back to depletion of PIP₂, the water-soluble, short-chain PIP₂ derivative, dioctanoyl-PIP₂ (hereafter, diC8-PIP₂), was added to the pipette solution in the conventional whole-cell configuration and measured Kir2.1 currents. Consistent with an essential role for PIP₂ in sustaining capillary Kir2.1 activity, inclusion of 10 μM diC8-PIP₂ largely abrogated the decline in Kir2.1 currents (FIG. 4B-4C). The initial current density (at t₀) was the same for the perforated-patch configuration and conventional whole-cell configuration dialyzed with or without Mg-ATP, or with diC8-PIP₂ and 0 mM Mg-ATP (FIG. 4D). The finding that diC8-PIP₂ did not elevate initial Kir2.1 currents suggests that these channels are saturated with PIP₂ under basal conditions.

Because replenishment of PIP₂ after depletion depends on PI4K and PIP5K activities and ATP hydrolysis (FIG. 4A), the effects of cell-permeable inhibitors of PIP₂ synthesis were tested on Kir2.1 currents recorded in the perforated-patch (intact-cytoplasm) configuration. The PI4K inhibitors PIK93 (300 nM) and phenylarsine oxide (10 μM) significantly suppressed Kir2.1 currents under conditions in which intracellular ATP was unperturbed; inhibition of PIP5K with UNC3230 (100 nM) yielded similar results (FIG. 4E-4F). These findings collectively indicate that ATP-dependent synthesis of PIP₂ is essential for sustained Kir2.1 activity in brain capillaries.

### Example 3—G_{q}PCR Stimulation Reduces Kir2.1 Currents by Decreasing PIP₂ Levels

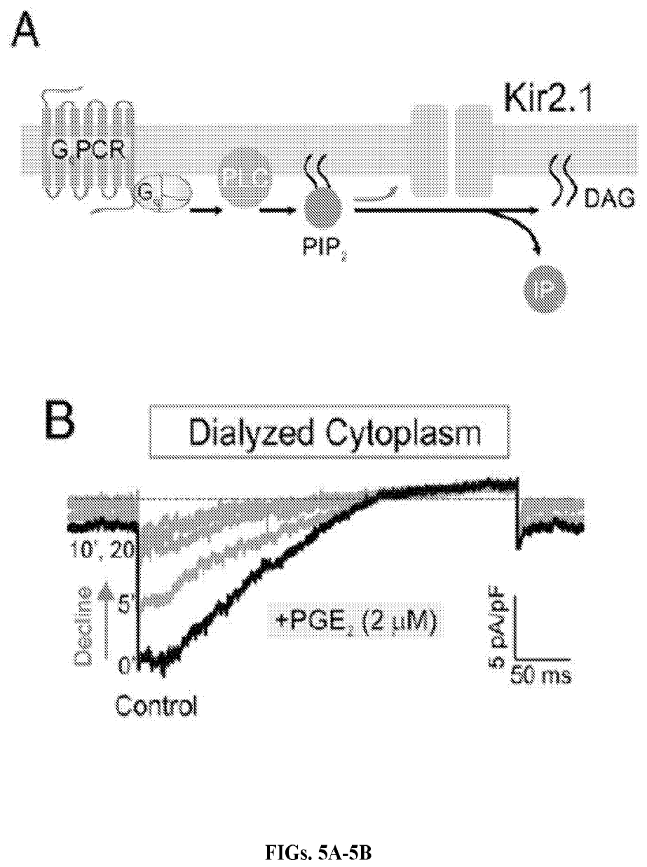

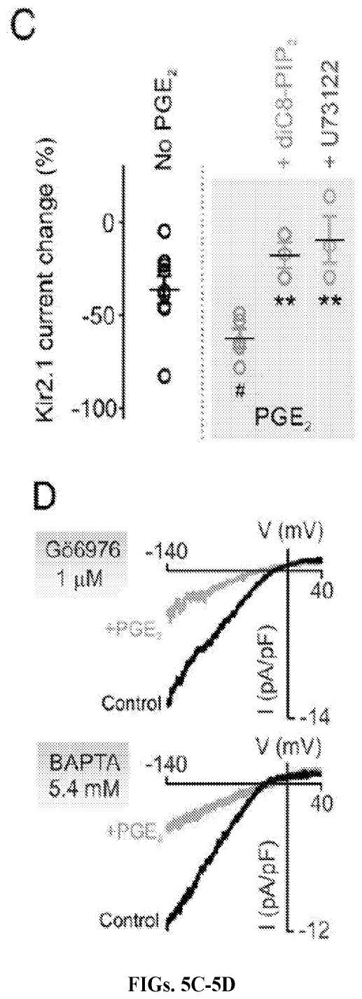

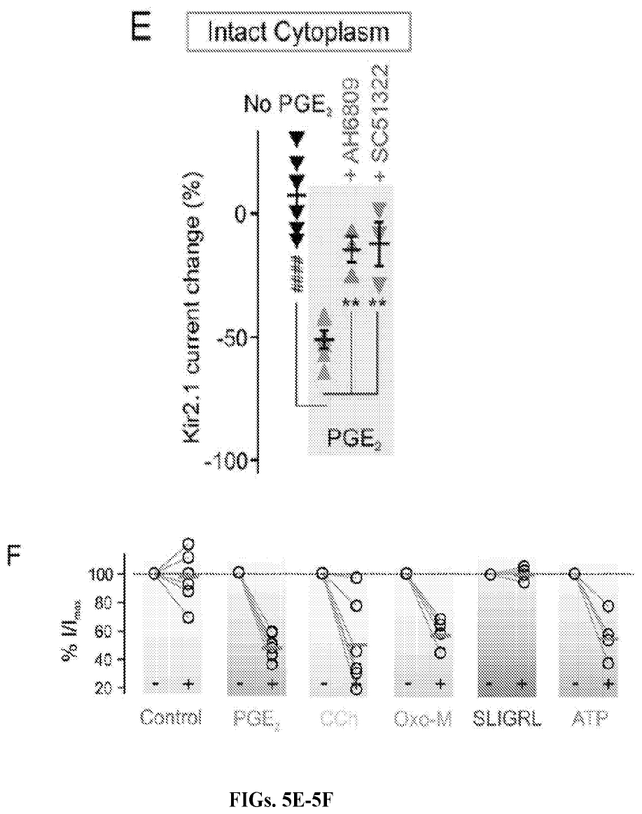

PIP₂ is key to the maintenance of functional inward-rectifier K+ channels, as indicated above (FIGS. 1A-1C and FIGS. 4A-4F) and reported previously (Huang et al., “Direct Activation of Inward Rectifier Potassium Channels by PIP₂ and its Stabilization by Gβγ,” Nature 391:803-806 (1998); D'Avanzo et al., “Direct and Specific Activation of Human Inward Rectifier K⁺ Channels by Membrane Phosphatidylinositol 4,5-bi-Sphosphate,” J Biol Chem 285:37129-37132 (2010); Hansen et al., “Structural Basis of PIP₂ Activation of the Classical Inward Rectifier K⁺ Channel Kir2.2,” Nature 477:495-498 (2011), which are hereby incorporated by reference in their entirety). Although PIP₂ is a minor phospholipid, it is nonetheless dynamic. Under physiological conditions, the primary driver of changes in PIP₂ levels is GqPCR-mediated activation of PLC and subsequent hydrolysis of PIP₂ to IP3 and diacylglycerol (FIG. 5A). A number of putative astrocyte-derived vasoactive substances implicated in neurovascular coupling, including PGE2 and ATP (Lacroix et al., “COX-2-Derived Prostaglandin E2 Produced by Pyramidal Neurons Contributes to Neurovascular Coupling in the Rodent Cerebral Cortex,” J Neurosci. 35:11791-11810 (2015); Zonta et al., “Neuron-to-Astrocyte Signaling is Central to the Dynamic Control of Brain Microcirculation,” Nat. Neurosci. 6:43-50 (2003); Wells et al., “A Critical Role for Purinergic Signaling in the Mechanisms Underlying Generation of BOLD fMRI Responses,” J Neurosci. 35:5284-5292 (2015); Kisler et al., “Cerebral Blood Flow Regulation and Neurovascular Dysfunction in Alzheimer Disease,” Nat Rev Neurosci 18:419-434 (2017), which are hereby incorporated by reference in their entirety), are GqPCR agonists; thus, their signaling is capable of promoting PLC-mediated PIP2 degradation. To determine whether activation of endothelial GqPCRs suppresses Kir2.1 channels via PIP₂ hydrolysis, Kir2.1 currents were examined in dialyzed capillary endothelial cells (no ATP in the patch pipette) following treatment with PGE2, which can signal through the prostanoid GqPCR, EP1 (Uekawa et al., “Obligatory Role of EP1 Receptors in the Increase in Cerebral Blood Flow Produced by Hypercapnia in the Mice,” PLoS One 11:e0163329 (2016); Dabertrand et al., “Prostaglandin E₂, a Postulated Astrocyte-Derived Neurovascular Coupling Agent, Constricts Rather than Dilates Parenchymal Arterioles,” J Cereb Blood Flow Metab 33:479-482 (2013), which are hereby incorporated by reference in their entirety). As shown in FIGS. 5B-5C, application of PGE2 (2 μM) to dialyzed cells accelerated the decay of Kir2.1 currents, almost doubling the extent of current decline after 15 minutes (62%), compared with that observed in matching time controls (36%) (FIG. 1C). Hindering PIP₂ synthesis through removal of Mg-ATP and enhancing its breakdown through activation of a GqPCR should decrease ambient PIP₂ levels and thus inhibit Kir2.1 channel activity. Accordingly, to calculate the time constant of Kir2.1 current decay (τ_{decay}), Kir2.1 currents were monitored over time following application of a PIP₂-depleting GqPCR agonist onto capillary endothelial cells dialyzed with 0 mM Mg-ATP. Using this experimental approach, a τ_{decay} of ˜7 to 13 minutes was estimated, which reflects the change in PIP₂ synthesis and breakdown. Note that, under these conditions, Kir2.1 current was not completely abolished (˜60 to 70% inhibition), suggesting residual ongoing PIP₂ synthesis. These slow decay kinetics (spanning minutes) are consistent with the high affinity of PIP₂ for Kir2.1 channels (Soom M, et al., “Multiple PIP₂ Binding Sites in Kir2.1 Inwardly Rectifying Potassium Channels,” FEBS Lett 490:49-53 (2001); Lopes CMB, et al., “Alterations in Conserved Kir Channel-PIP₂ Interactions Underlie Channelopathies,” Neuron 34:933-944 (2002); Du et al., “Characteristic Interactions with Phosphatidylinositol 4,5-bi-Sphosphate Determine Regulation of Kir Channels by Diverse Modulators,” J Blot Chem 279:37271-37281 (2004); Kruse et al., “Regulation of Voltage-Gated Potassium Channels by PI(4,5)P₂,” J Gen Physiol 140:189-205 (2012), which are hereby incorporated by reference in their entirety).

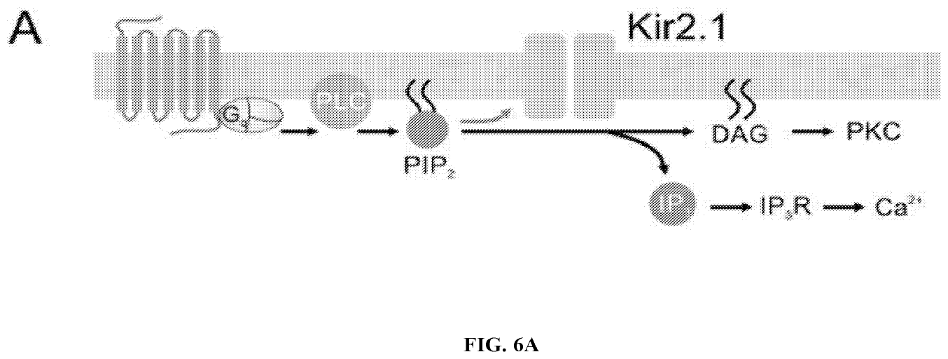

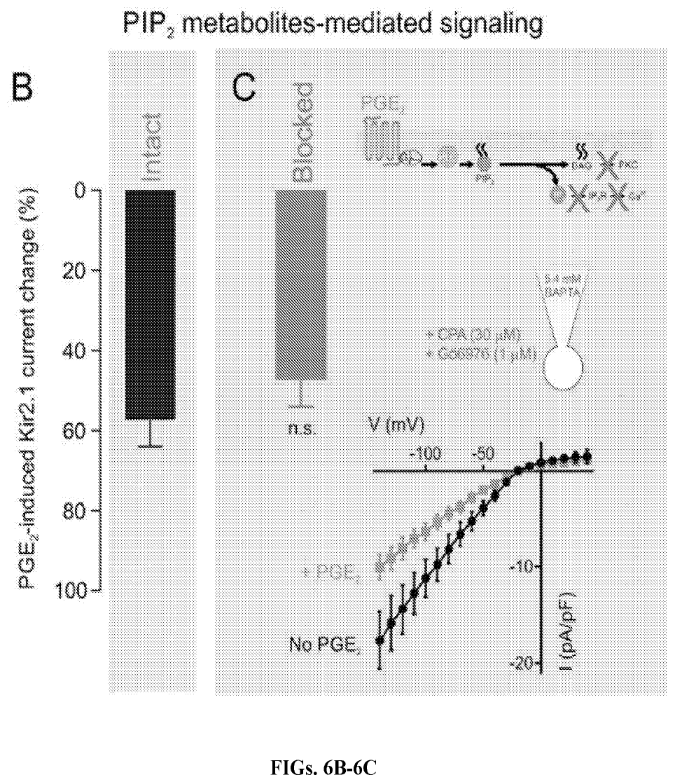

Introduction of diC8-PIP2 (10 μM) into the cytosol or inhibition of PLC with U73122 (10 μM) are interventions that serve to compensate for or prevent PLC-dependent PIP₂ degradation, respectively. Both maneuvers completely abrogated the PGE2-induced reduction in Kir2.1 current (FIG. 5C), confirming the involvement of PIP₂ hydrolysis downstream of activation of the GqPCR-PLC pathway in the decay of Kir2.1 activity. The effect of PIP₂ hydrolysis on Kir2.1 channel activity was not attributable to the engagement of signaling pathways mediated by the PIP₂ breakdown products IP3 or diacylglycerol. Neither rapid chelation of cytoplasmic Ca²⁺ with intracellular 1,2-bis(2-aminophenoxy)ethane-N,N,N′,N′-tetraacetic acid (BAPTA) (5.4 mM) nor inhibition of protein kinase C with 12-(2-cyanoethyl)-6,7,12,13-tetrahydro-13-methyl-5-oxo-5H-indolo(2,3-a)pyrrolo(3,4-c)-carbazole (Gö6976) (1 μM) attenuated the PGE2-mediated suppression of Kir2.1 currents (FIG. 5D). Along the same lines, simultaneous blockade of both diacylglycerol-PKC and IP3-IP3R-Ca²⁺ signaling cascades failed to impact the inhibitory effect of PGE2 on Kir2.1 current in dialyzed capillary endothelial cells (FIGS. 6A-6C). Taken together, these data show that PGE2 acts through GqPCR activation to stimulate PLC and decrease PIP₂ levels, thereby deactivating Kir2.1 channels independently of PIP₂ metabolites.

An important confirmation of this conclusion was provided by experiments performed in cytoplasm-intact mode (perforated patch), in which endogenous ATP and PIP₂ are not perturbed and Kir2.1 currents were found to be resistant to decline (FIG. 1A and FIG. 5E). These experiments showed that application of the GqPCR agonist PGE2 rapidly (onset, <60 s) and dramatically reduced Kir2.1 currents (˜51% decline) (FIG. 5E), consistent with the idea that GqPCR stimulation exerts an inhibitory effect on Kir2.1 channel activity. The inhibitory effect of PGE2 was prevented by the nonselective prostanoid receptor (EP1/EP2/EP3) antagonist AH6809 (10 μM) and, notably, by the selective EP1 antagonist SC51322 (1 μM), suggesting that PGE2 acts through the Gq-coupled EP1 receptor to inhibit capillary Kir2.1 channel activity (FIG. 5E).

To assess the generalizability of this mechanism, changes in Kir2.1 currents induced by PGE2 were compared with those induced by muscarinic receptor agonists, the protease-activated receptor-2 (PAR2) agonist SLIGRL-NH2, and the purinergic receptor agonist ATP, all of which are capable of signaling through GqPCRs. Using capillary endothelial cells in the cytoplasm-intact mode (perforated patch), it was found that the muscarinic receptor agonists carbachol and N,N,N-trimethyl-4-(2-oxo-1-pyrolidinyl)-2-butyn-1-ammonium iodide (oxotremorine M) (10 μM each) and purinergic receptor agonist ATP (30 μM) decreased Kir2.1 currents by 48±12%, 40±5%, and 43±8%, respectively, after a 15-minute incubation. These effects were comparable with those induced by PGE2 (51±4%) under similar experimental conditions (FIG. 5F). Interestingly, although SLIGRL-NH2 has been shown to cause endothelial-dependent dilation of surface cerebral arteries (McNeish et al., “Possible Role for K⁺ in Endothelium-Derived Hyperpolarizing Factor-Linked Dilatation in Rat Middle Cerebral Artery,” Stroke 36:1526-1532 (2005), which is hereby incorporated by reference in its entirety), this PAR2 agonist (5 μM) had no effect on capillary Kir2.1 currents (FIG. 5F), possibly reflecting rapid receptor desensitization and a rebound in PIP₂ levels following activation (Jung et al., “Contributions of Protein Kinases and β-Arrestin to Termination of Protease-Activated Receptor 2 Signaling,” J Gen Physiol 147:255-271 (2016), which is hereby incorporated by reference in its entirety). It is also possible that differences in receptor expression levels, requirements for specific localization patterns, and/or differential GqPCR-dependent mobilization of PIP₂ contributes to GqPCR agonist efficacy (Dickson et al., “Quantitative Properties and Receptor Reserve of the IP₃ and Calcium Branch of G_{q}-Coupled Receptor Signaling,” J Gen Physiol 141:521-535 (2013); Cho et al., “Receptor-Induced Depletion of Phosphatidylinositol 4,5-Bisphosphate Inhibits Inwardly Rectifying K⁺ Channels in a Receptor-Specific Manner,” Proc Natl Acad Sci USA 102:4643-4648 (2005); Cho et al., “Low Mobility of Phosphatidylinositol 4,5-Bisphosphate Underlies Receptor Specificity of Gq-Mediated Ion Channel Regulation in Atrial Myocytes,” Proc Natl Acad Sci USA 102:15241-15246 (2005), which are hereby incorporated by reference in their entirety).

### Example 4—GqPCR Stimulation Suppresses Capillary-to-Arteriole Electrical Signaling

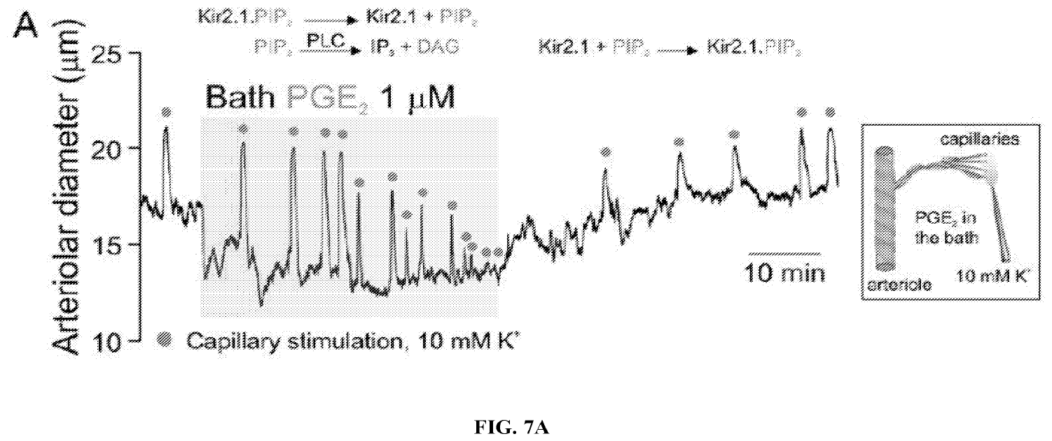

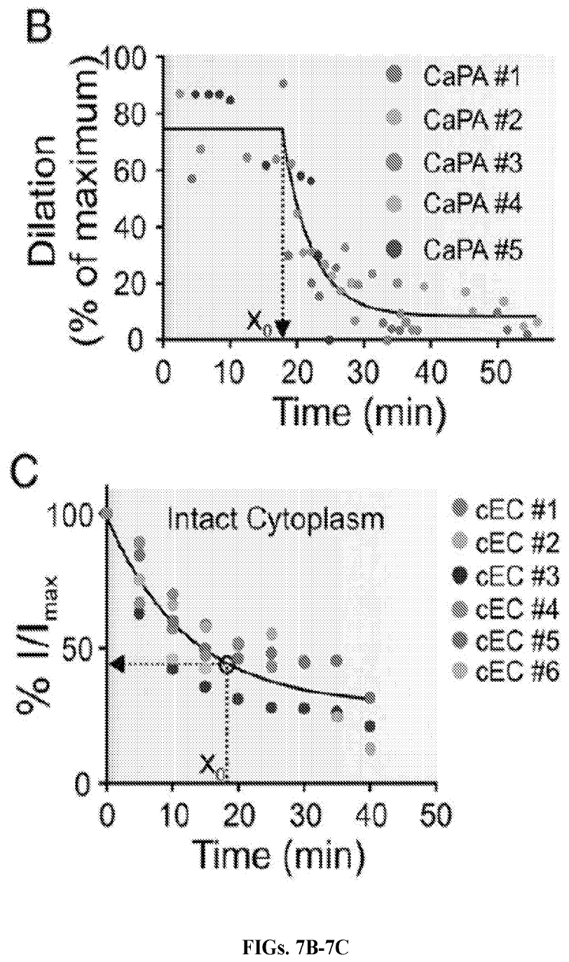

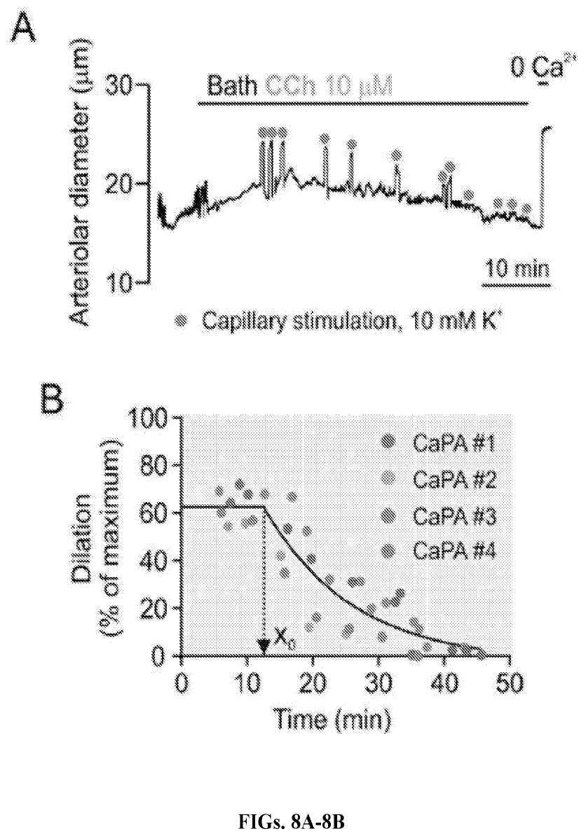

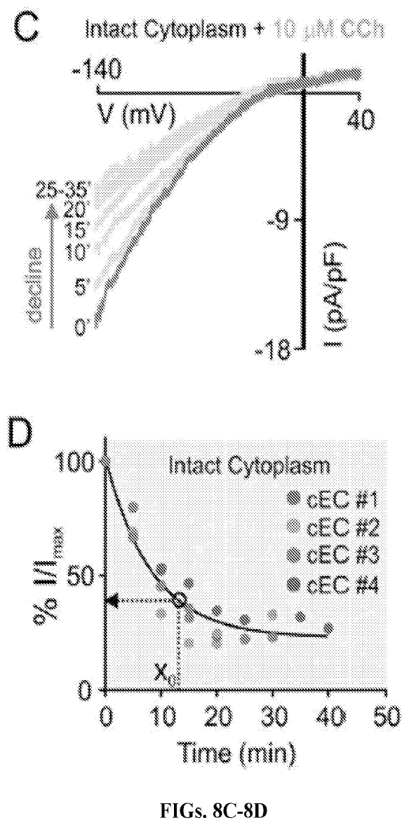

Capillary Kir2.1 channels sense increases in [K⁺]ₒ caused by increased neuronal activity and initiate a hyperpolarizing signal. By virtue of strong electrical coupling between endothelial cells, retrograde hyperpolarization ascends to upstream feeding arterioles to enhance cerebral blood flow to the site of signal initiation (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety). The fact that GqPCR activation suppresses Kir2.1 currents in capillary endothelial cells (FIGS. 5A-5F) suggests that GqPCR agonists could alter capillary-to-arteriole signaling and ensuing changes in blood flow. To investigate this possibility, the recently developed ex vivo capillary-parenchymal arteriole (CaPA) preparation was used, which makes it possible to monitor effects of local stimulation of capillary branches on upstream arteriolar diameter in a reduced environment (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety). Focal stimulation of capillaries in the CaPA preparation with 10 mM K⁺ induced a reproducible dilatory response in the attached arteriolar segment (FIG. 7A), reflecting activation of capillary Kir2.1 channels (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety). To test the influence of GqPCR signaling on Kir2.1-mediated capillary-to-arteriole signaling, the postulated neurovascular coupling agent PGE2 (1 μM) was bath-applied to globally activate EP1 receptors and degrade PIP₂. Consistent with PIP₂ breakdown and disabling of Kir2.1 channels, PGE2 gradually attenuated and, ultimately, abolished K⁺-induced upstream vasodilation (FIG. 7A). Capillary Kir2.1-mediated upstream arteriolar dilation was similarly suppressed by the muscarinic receptor agonist carbachol (FIGS. 8A-8D). Capillary responsiveness to elevated external K recovered after removal of PGE2 from the capillary-parenchymal arteriole preparation (τ_{recovery}≈17 minutes) (FIG. 7A). The latter observation is consistent with the idea that the PIP₂ necessary for Kir2.1 channel activity was replenished during the period between PGE2 washout and subsequent remeasurement. Notably, there was a lag phase (X0≈18 minutes) between PGE2 application and onset of the inhibition of capillary-mediated arteriolar dilation (FIG. 7B). During this lag period, Kir2.1 currents recorded in the perforated-patch configuration declined steadily (τ_{decay}≈12 minutes), but K⁺-mediated retrograde dilatory signaling remained intact until Kir2.1 currents reached ˜50% of their maximal amplitude (FIG. 7C). These observations suggest that a critical number of Kir2.1 channels must deactivate to impact the regenerative propagation of hyperpolarization from capillaries to the upstream arteriole.

### Example 5—In Vivo G_{q}PCR Stimulation Inhibits K⁺-Evoked Capillary Hyperemia

Raising [K¹]ₒ around capillaries in vivo evokes upstream arteriolar dilation and increases capillary RBC flux (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety). Stimulation of GqPCRs inhibits Kir2.1 channels and capillary-to-arteriole signaling in the ex vivo capillary-parenchymal arteriole preparation (FIGS. 5A-5F, 7A-7C, and 8A-8D). Building on these results, it was sought to determine whether activation of endothelial cell GqPCRs by systemic administration of a suitable agonist alters responses to elevated [K¹]ₒ in vivo, measured by imaging RBC flux in mice using a cranial window model. For these experiments, carbachol was chosen, which exerted inhibitory effects on capillary Kir2.1 currents (FIGS. 5A-5F and FIGS. 8A-8D) and Kir2.1-mediated capillary-to-arteriole signaling (FIGS. 8A-8D) similar to those evoked by PGE2. The rationale for using carbachol over PGE2 during in vivo imaging is multifold. First, carbachol is a positively charged choline carbamate with a characteristically lipophobic structure. Carbachol is thus unable to cross the blood-brain barrier (BBB), a property that is key to the experimental goal of influencing brain endothelial cells without directly affecting other brain cells. In contrast, prostaglandins are highly lipophilic; PGE2, in particular, crosses the BBB (Jones et al., “PGE2 in the Perinatal Brain: Local Synthesis and Transfer Across the Blood Brain Barrier,” J. Lipid Mediat. 6:487-492 (1993), which is hereby incorporated by reference in its entirety) and can contribute to pathological BBB breakdown (Schmidley et al., “Brain Tissue Injury and Blood-Brain Barrier Opening Induced by Injection of LGE₂ or PGE₂,” Prostaglandins Leukot. Essent. Fatty Acids 47:105-110 (1992), which is hereby incorporated by reference in its entirety). Second, PGE2, which can be synthesized in the brain endothelium (Wilhelms et al., “Deletion of Prostaglandin E₂ Synthesizing Enzymes in Brain Endothelial Cells Attenuates Inflammatory Fever,” J. Neurosci. 34:11684-11690 (2014), which is hereby incorporated by reference in its entirety), is highly pyrogenic and exerts proinflammatory actions through multiple effects on different cell types (Saper CB “Neurobiological Basis of Fever,” Ann. NY Acad. Sci. 856:90-94 (1998); Nakanishi et al., “Multifaceted Roles of PGE₂ in Inflammation and Cancer,” Semin. Immunopathol. 35:123-137 (2013), which are hereby incorporated by reference in their entirety). Third, PGE2 evokes mixed vasomotor effects that may interfere with the question of interest: for example, constricting isolated brain parenchymal arterioles, as previously reported (Dabertrand et al., “Prostaglandin E₂, a Postulated Astrocyte-Derived Neurovascular Coupling Agent, Constricts Rather than Dilates Parenchymal Arterioles,” J. Cereb. Blood Flow Metab. 33:479-482 (2013), which is hereby incorporated by reference in its entirety), but dilating other vascular beds, as reported by others (Zonta et al., “Neuron-to-Astrocyte Signaling is Central to the Dynamic Control of Brain Microcirculation,” Nat. Neurosci. 6:43-50 (2003); Ellis et al., “Vasodilation of Cat Cerebral Arterioles by Prostaglandins D₂, E₂, G₂, and I₂,” Am. J. Physiol. 237:H381-H385 (1979); Takano et al., “Astrocyte-Mediated Control of Cerebral Blood Flow,” Nat. Neurosci. 9:260-267 (2006), which are hereby incorporated by reference in their entirety). Such mixed vasomotor effects can lead to alterations in blood pressure and could thus introduce a confounding factor to in vivo experiments. Carbachol, in contrast, minimally altered parenchymal arteriolar diameter (FIGS. 8A-8D), and, at the lower systemic dosage employed here, has no effect on arterial blood pressure or partial pressures of O₂ or CO₂ in the blood (Aubineau et al., “Parasympathomimetic Influence of Carbachol on Local Cerebral Blood Flow in the Rabbit by a Direct Vasodilator Action and an Inhibition of the Sympathetic-Mediated Vasoconstriction,” Br. J. Pharmacol. 68:449-459 (1980), which is hereby incorporated by reference in its entirety).

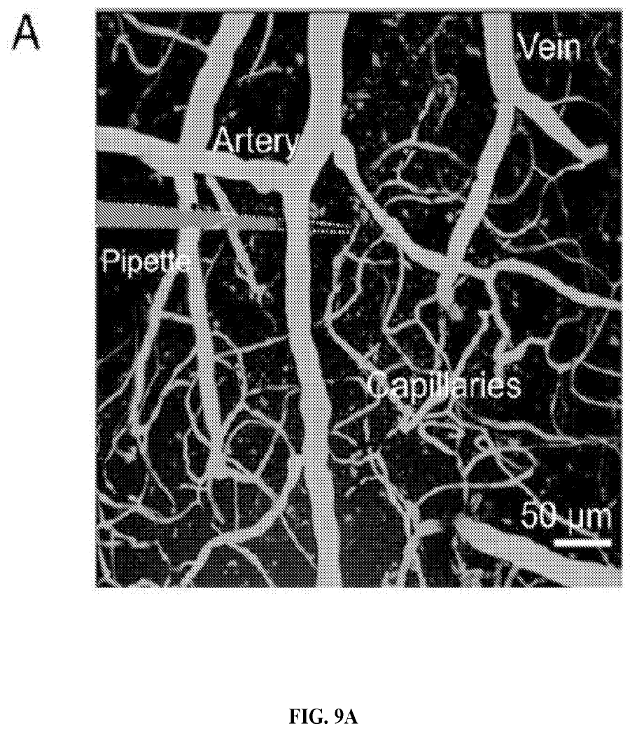

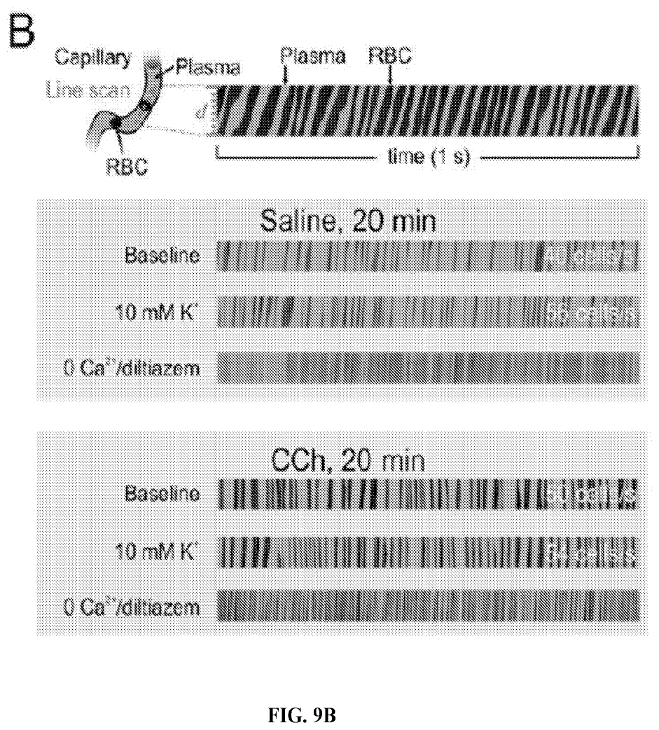

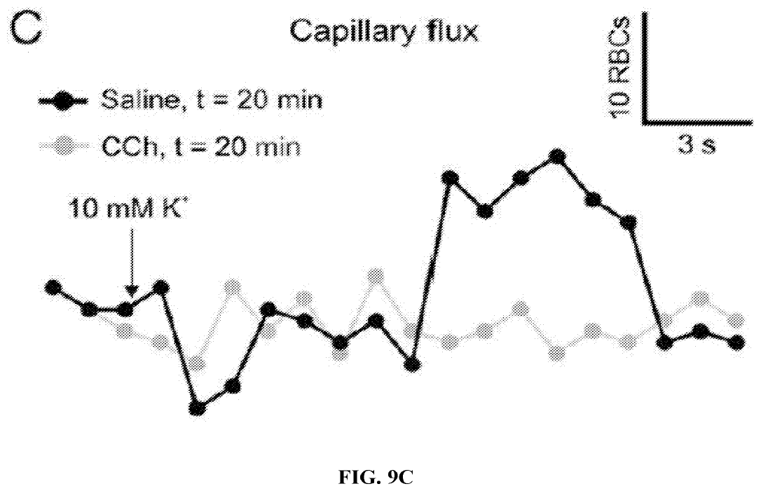

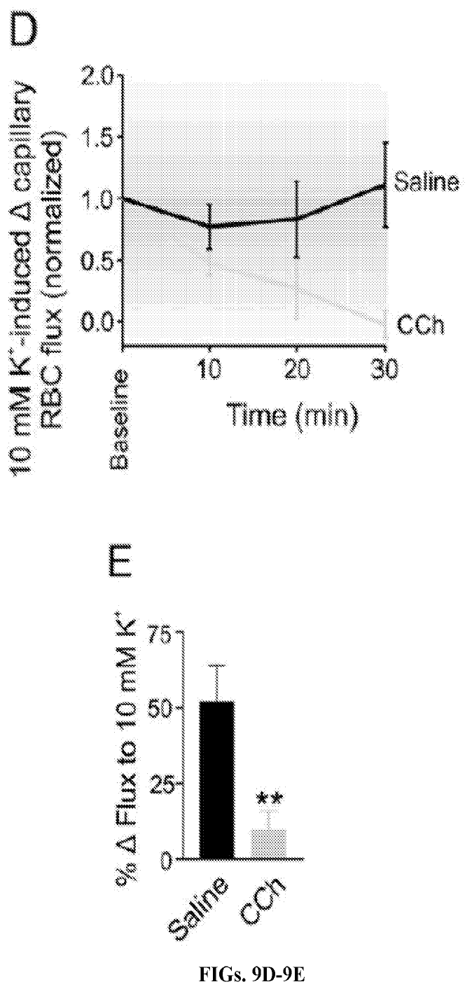

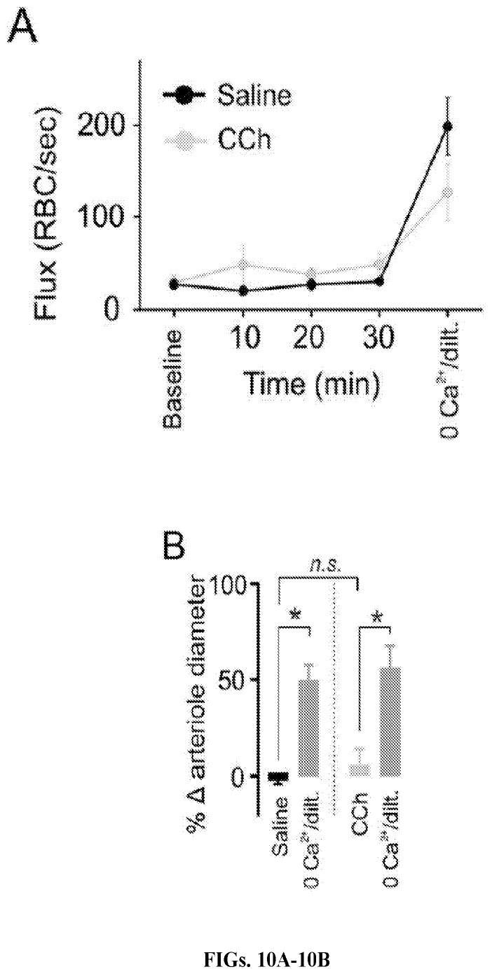

Anesthetized mice were fitted with a cranial window and systemically injected with fluorescein isothiocyanate (FITC)-labeled dextran to allow visualization of the vascular network and support contrast imaging of RBCs by two-photon laser-scanning microscopy (FIG. 9A). Mice were divided into two experimental groups: saline-treated (time-control) and carbachol-treated. Mice in the carbachol-treated group were systemically administered a low dose (0.6 μg/kg body weight) of carbachol via intravascular injection into the retroorbital venous sinus to activate endothelial muscarinic GqPCRs. Mice in the control group were similarly administered saline. K⁺-evoked, Kir2.1-mediated hyperemia was investigated in both groups before (baseline) and 10, 20, and 30 minutes after injection. Focal stimulation of a brain capillary in control mice by pressure ejection (300 ms) of 10 mM K⁺ via a micropipette evoked a rapid increase (52±12% at t=20 minutes post-saline administration) in capillary RBC flux in the stimulated segment (FIG. 9B-9C). As predicted based on ex vivo results, circulating carbachol profoundly decreased the in vivo response to 10 mM K⁺, yielding a K⁺-induced increase in RBC flux (10±6% at t=20 minutes after carbachol injection) more than fivefold lower than that in controls (FIG. 9B-9E). Baseline capillary RBC flux (before K⁺ application) did not change in the carbachol-injected group over the course of 30 minutes (FIG. 10A). The diameters of parenchymal arterioles upstream of the tested capillary segments were not changed by a 20-minute carbachol treatment compared with that in the saline (time-control) group (FIG. 10B). At the conclusion of each 30-minute experiment, application of a 0-mM Ca²⁺ solution containing 200 μM diltiazem (included to inhibit arterial/arteriolar Ca²⁺ channels) to the cranial surface dramatically dilated arterioles and enhanced capillary RBC flux in both saline- and carbachol-treated groups (FIG. 9C and FIG. 10). This latter observation is important, because it indicates that vasodilatory and RBC flux response are not already maximal, confirming that the lack of a hyperemic response to external K⁺ postcarbachol treatment is attributable to Kir2.1 channel deactivation.

### Discussion of Examples 1-5

Capillary endothelial cells in the brain are anatomically positioned to sense neuronal activity and orchestrate the matching of cerebral blood flow to the moment-to-moment metabolic demands of the brain. They are also equipped with the molecular machinery—Kir2.1 channels and GqPCRs—necessary to respond to factors—K⁺ and GqPCR agonists—that have been implicated in neurovascular coupling. It has been recently reported that Kir2.1 channels in brain capillary endothelial cells function as K⁺ sensors. Increases in [K⁺]ₒ associated with neuronal activity trigger an ascending hyperpolarizing signal that dilates upstream arterioles and enhances capillary RBC flux and cerebral blood flow (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety). The present study sheds light on the molecular features that regulate this electrical signaling. Specifically, the results show that PIP₂ levels are critical determinants in sustaining Kir2.1 channel activity in the brain capillary endothelium, supporting the concept that this phosphoinositide plays a central role in regulating Kir2.1 channel-mediated electrical signaling during neurovascular coupling. This concept is extended and provides strong evidence for the existence of communication from GqPCRs to this electrical signaling mechanism, reflecting the dependence of Kir2.1 channel structure and function on cellular PIP₂ and the ability of GqPCRs to deplete it. Importantly, it is further shown that GqPCR stimulation short-circuits the ascending electrical signal originating at the capillary level and abrogates upstream dilation, both ex vivo (FIG. 7) and in vivo (FIG. 9). This paradigm establishes PIP₂ as a point of intersection between GqPCR-mediated signaling and electrical signaling. This model uniquely highlights the role of GqPCRs as a signaling “switch” with the potential to determine the extent and directionality of the electrical signaling modality in brain capillaries and ultimately modulate functional hyperemic responses.

PIP₂ has been shown to bind to and modulate a plethora of ion channels, including members of the Kir2 channel family (Hille et al., “Phosphoinositides Regulate Ion Channels,” Biochim. Biophys. Acta 1851:844-856 (2015), which is hereby incorporated by reference in its entirety). An important feature of PIP₂ is that its cellular levels are dynamically regulated through continuous synthesis by lipid kinases and breakdown by lipases. PIP₂ is synthesized by the lipid kinases PI4K and PIP5K, which convert PI to PIP and PIP to PIP₂, respectively. This process is highly ATP concentration-dependent, reflecting the relatively low ATP affinity of these lipid kinases (Knight et al., “Features of Selective Kinase Inhibitors,” Chem. Biol. 12: 621-637 (2005); Suer et al., “Human Phosphatidylinositol 4-Kinase Isoform PI4K92. Expression of the Recombinant Enzyme and Determination of Multiple Phosphorylation Sites,” Eur. J. Biochem. 268:2099-2106 (2001); Balla et al., “Phosphatidylinositol 4-Kinases: Old Enzymes with Emerging Functions,” Trends Cell Biol. 16:351-361 (2006), which are hereby incorporated by reference in their entirety). Consistent with this, the results indicate that sustaining the PIP₂ levels necessary to support Kir2.1 channel activity is critically dependent on the intracellular concentration of ATP. On the breakdown side of this equation, PLC, activated in response to stimulation of GqPCRs, hydrolyzes PIP₂ to IP3 and diacylglycerol. It has been shown that GqPCR-mediated depletion of PIP₂ is capable of altering the activity of PIP₂-regulated channels (Kobrinsky et al., “Receptor-Mediated Hydrolysis of Plasma Membrane Messenger PIP₂ Leads to K⁺-Current Desensitization,” Nat. Cell Biol. 2:507-514 (2000), which is hereby incorporated by reference in its entirety), suggesting that persistent depletion of this minor (˜1%) plasma membrane phospholipid in capillary endothelial cells would have major consequences for Kir2.1 activity. Indeed, it was found that multiple GqPCR agonists, including those implicated in neurovascular coupling (PGE2 and ATP) (Lacroix et al., “COX-2-Derived Prostaglandin E2 Produced by Pyramidal Neurons Contributes to Neurovascular Coupling in the Rodent Cerebral Cortex,” J. Neurosci. 35:11791-11810 (2015); Zonta et al., “Neuron-to-Astrocyte Signaling is Central to the Dynamic Control of Brain Microcirculation,” Nat. Neurosci. 6:43-50 (2003); Wells et al., “A Critical Role for Purinergic Signaling in the Mechanisms Underlying Generation of BOLD fMRI Responses,” J. Neurosci. 35:5284-5292 (2015); Kisler et al., “Cerebral Blood Flow Regulation and Neurovascular Dysfunction in Alzheimer Disease,” Nat. Rev. Neurosci. 18:419-434 (2017), which are hereby incorporated by reference in their entirety), are capable of deactivating Kir2.1 currents (FIG. 5). These data also confirmed that the ability of GqPCR agonists to suppress capillary Kir2.1 channel activity in the capillary endothelium is not attributable to IP3-IP3R-Ca²⁺ or diacylglycerol-PKC signaling (FIG. 5 and FIG. 6). Notably, enhanced GqPCR/PLC activation can promote PIP₂ breakdown at rates that exceed ongoing synthesis (FIG. 5E-5F). The differential kinetics of PIP₂ hydrolysis and repletion align with previous direct in vitro measurements, as well as in silico calculations (Dickson et al., “Quantitative Properties and Receptor Reserve of the IP₃ and Calcium Branch of G_{q}-Coupled Receptor Signaling,” J. Gen. Physiol. 141:521-535 (2013), which is hereby incorporated by reference in its entirety), and are important when considering the long-lasting effects of endogenous GqPCR agonists.

The electrophysiological experiments illustrate that initial Kir2.1 channel activity was similar in dialyzed capillary endothelial cells, with or without PIP₂ supplementation (FIG. 4), implying that Kir2.1 channels are saturated with PIP₂ under basal conditions. These findings are consistent with structural studies of Kir2 channels, including reports of the crystal structure of the Kir2.2 channel (Hansen et al., “Structural Basis of PIP₂ Activation of the Classical Inward Rectifier K⁺ Channel Kir2.2,” Nature 477:495-498 (2011), which is hereby incorporated by reference in its entirety), which have collectively established that these channels require PIP₂ binding to maintain their active conformation (D'Avanzo et al., “Direct and Specific Activation of Human Inward Rectifier K⁺ Channels by Membrane Phosphatidylinositol 4,5-bi-Sphosphate,” J. Biol. Chem. 285:37129-37132 (2010), which is hereby incorporated by reference in its entirety). In keeping with the reported high PIP₂-Kir2.1 affinity and/or specificity (D'Avanzo et al., “Direct and Specific Activation of Human Inward Rectifier K⁺ Channels by Membrane Phosphatidylinositol 4,5-bi-Sphosphate,” J. Biol. Chem. 285:37129-37132 (2010); Du et al., “Characteristic Interactions with Phosphatidylinositol 4,5-bi-Sphosphate Determine Regulation of Kir Channels by Diverse Modulators,” J. Biol. Chem. 279:37271-37281 (2004); D'Avanzo et al., “Energetics and Location of Phosphoinositide Binding in Human Kir2.1 Channels,” J. Biol. Chem. 288:16726-16737 (2013), which are hereby incorporated by reference in their entirety), it was found that the kinetics of capillary Kir2.1 channel deactivation following GqPCR activation or lowering of intracellular ATP levels are slow, consistent with high affinity binding. Nonetheless, the data clearly indicate that sustained GqPCR activation is capable of causing sufficient PIP₂ dissociation to deactivate Kir2.1 channels.

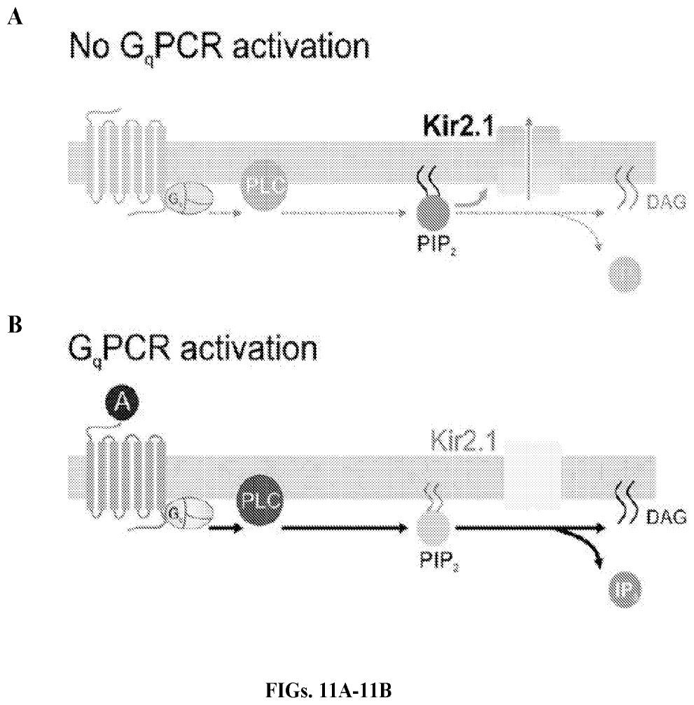

The slow kinetics of Kir2.1 channel inhibition and the corresponding requirement for sustained GqPCR activation to deplete PIP₂ sufficiently to deactivate the channel raise questions about the circumstances under which capillaries would experience prolonged exposure to receptor agonist. Given that brain capillaries are positioned in close proximity to all neurons and astrocytes (Blinder et al., “The Cortical Angiome: An Interconnected Vascular Network with Noncolumnar Patterns of Blood Flow,” Nat. Neurosci. 16:889-897 (2013); Shih et al, “Robust and Fragile Aspects of Cortical Blood Flow in Relation to the Underlying Angioarchitecture,” Microcirculation 22:204-218 (2015), which are hereby incorporated by reference in their entirety), capillaries are presumably exposed to a microenvironment containing potential physiological stimuli, including varying concentrations of GqPCR agonists postulated to serve as neurovascular coupling agents. Moreover, rates of receptor-mediated PIP₂ breakdown exceed those of PIP₂ resynthesis, indicating that such GqPCR agonists could trigger an extended decline in PIP₂ levels (Dickson et al., “Quantitative Properties and Receptor Reserve of the IP₃ and Calcium Branch of G_{q}-Coupled Receptor Signaling,” J. Gen. Physiol. 141:521-535 (2013), which is hereby incorporated by reference in its entirety). Viewed from this perspective, GqPCR-mediated PIP₂ depletion represents a potential entry point for local microenvironmental influences to dampen capillary Kir2.1-mediated electrical signaling (FIGS. 11A-11B). GqPCR signaling is also associated with initiation of an intracellular Ca²⁺ signal, reflecting IP3 generation and Ca²⁺ release from intracellular stores. This suggests that astrocyte- and/or neuron-derived agonists implicated in neurovascular coupling could also engage a Ca²⁺ signaling-based mechanism in capillary endothelial cells. It is thus conceivable that, in addition to setting the gain of electrical signaling in brain capillaries, activation of capillary GqPCRs by putative neurovascular coupling agents might also initiate a Ca²⁺ signal that could play a role in functional hyperemia.

Intriguingly, experiments using the capillary-parenchymal arteriole preparation showed that GqPCR activation inhibited capillary Kir2.1-mediated upstream arteriolar dilation only after a lag phase, during which Kir2.1 currents, measured in isolated endothelial cells, steadily declined. An electrophysiological analysis of endothelial cells using the intact-cytoplasm configuration showed that the duration of this lag phase corresponded to the time required for deactivation of ˜50% of Kir2.1 channels. These observations suggest that there is a minimum Kir2.1 channel density below which retrograde electrical signaling cannot occur. There are two conceptual scenarios in which the existence of such a threshold in Kir2.1 channel number could come into play. First, the originating endothelial cells may not move toward the K⁺ equilibrium potential (E_{K}) upon exposure to elevated [K⁺]— a requirement for initiating propagating hyperpolarization—if outward current through Kir2.1 channels is below a critical level. Alternatively, distant capillary endothelial cells may be unable to support the regenerative propagation of hyperpolarization if Kir2.1 current falls below a certain point. Experimental and computational modeling investigations are required to determine which scenario more accurately describes GqPCR-induced suppression of capillary electrical signaling.

One implication of the ATP concentration-dependent synthesis of PIP₂ is that modest decreases in ATP that would have no effect on high ATP affinity cellular reactions could compromise ongoing phosphoinositide repletion. In certain pathological settings, energy production is compromised, and cellular ATP levels in the brain decrease. Cerebral ischemia, for example, triggers a profound drop in [ATP]ᵢ (Kawauchi et al., “Light Scattering Change Precedes Loss of Cerebral Adenosine Triphosphate in a Rat Global Ischemic Brain Model,” Neurosci. Lett. 459:152-156 (2009); Matsunaga et al., “Energy-Dependent Redox State of Heme a+a₃ and Copper of Cytochrome Oxidase in Perfused Rat Brain In Situ,” Am. J. Physiol. 275:C1022-C1030 (1998), which are hereby incorporated by reference in their entirety), which would be expected to suppress electrical signaling through Kir2.1 channels. Another example is cortical spreading depression, in which a slow wave of depolarization propagates across the cerebral cortex. This wave is associated with decreased glucose and ATP levels, along with global neurotransmitter release and, presumably, subsequent GqPCR activation (Ayata et al., “Spreading Depression, Spreading Depolarizations, and the Cerebral Vasculature,” Physiol. Rev. 95:953-993 (2015), which is hereby incorporated by reference in its entirety). These latter observations offer alternative avenues for PIP₂ depletion through changes in the brain metabolic status; whether this will affect capillary signaling awaits confirmation.

Collectively, the results presented here provide strong evidence for a novel paradigm in which PIP₂ is a central player in the regulation of capillary endothelial signaling. Maintaining sufficient PIP₂ levels ensures proper capillary-to-arteriole electrical signaling whereas physiological or pathological decreases in the levels of this phospholipid would determine the strength and extent of this signaling, thereby impacting cerebral blood flow.

### Materials and Methods for Examples 6-10

Animal models. The transgenic (Tg) mouse lines, TgNotch3^{WT} and TgNotch3^{R169C}, have been previously described (Dabertrand et al., “Potassium Channelopathy-like Defect Underlies Early-stage Cerebrovascular Dysfunction in a Genetic Model of Small Vessel Disease,” Proc. Nat'l. Acad. Sci. 112(7):E796-805 (2015), which is hereby incorporated by reference in its entirety). Non-Tg mice are non-transgenic littermates obtained during breeding of TgNotch3^{WT} and TgNotch3^{R169C} mice, and were used as wild-type mice. 6 month-old animals were euthanized by intraperitoneal injection of sodium pentobarbital (100 mg/kg) followed by rapid decapitation. Mice were used at this age because this is well in advance (6 months) of the development of significant white matter lesion burden, and for the sake of comparison with previous studies (Joutel et al., “Cerebrovascular Dysfunction and Microcirculation Rarefaction Precede White Matter Lesions in a Mouse Genetic Model of Cerebral Ischemic Small Vessel Disease,” JCI 120:433-435 (2010), which is hereby incorporated by reference in its entirety). TgNotch3^{WT} and TgNotch3^{R169C} mice (on an FVB/N background) overexpress rat wild-type NOTCH3 and the CADASIL-causing NOTCH3(R169C) mutant protein, respectively, to a similar degree (˜4-fold) compared with the levels of endogenous NOTCH3 in Non-Tg mice (Joutel et al., “Cerebrovascular Dysfunction and Microcirculation Rarefaction Precede White Matter Lesions in a Mouse Genetic Model of Cerebral Ischemic Small Vessel Disease,” JCI 120:433-435 (2010); Cognat et al., “Early White Matter Changes in CADASIL: Evidence of Segmental Intramyelinic Oedema in a Pre-Clinical Mouse Model,” Acta Neuropathol. Commun. 2:49 (2014), which are hereby incorporated by reference in their entirety). Expression of CADASIL-causing mutations at normal endogenous levels does not produce a CADASIL-like phenotype, likely because the slowly developing mutant phenotype is unable to manifest during the short lifespan of a mouse (Joutel et al., “Cerebrovascular Dysfunction and Microcirculation Rarefaction Precede White Matter Lesions in a Mouse Genetic Model of Cerebral Ischemic Small Vessel Disease,” JCI 120:433-435 (2010), which is hereby incorporated by reference in its entirety). Overexpression of the mutant protein overcomes this constraint and is thus a key feature of this model. All experimental protocols used in this study were in accord with institutional guidelines approved by the Institutional Animal Care and Use Committee of the University of Vermont.

Capillary endothelial cell isolation. Single capillary endothelial cells (cECs) were obtained from mouse brains by mechanical disruption of two 160-μm-thick brain slices using a Dounce homogenizer, as previously described (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety). Slices were homogenized in ice-cold artificial cerebrospinal fluid, with the composition 124 mM NaCl, 3 mM KCl, 2 mM CaCl₂, 2 mM MgCl₂, 1.25 mM NaH₂PO₄, 26 mM NaHCO₃, and 4 mM glucose. Debris were removed by passing the homogenate through a 62-μm nylon mesh. Retained capillary fragments were washed into dissociation solution composed of 55 mM NaCl, 80 mM Na-glutamate, 5.6 mM KCl, 2 mM MgCl₂, 4 mM glucose, and 10 mM HEPES (pH 7.3) containing neutral protease (0.5 mg/ml), elastase (0.5 mg/ml; Worthington, USA) and 100 μM CaCl₂, and incubated for 24 minutes at 37° C. Following this step, 0.5 mg/ml collagenase type I (Worthington, USA) was added and the solution was incubated for an additional 2 minutes at 37° C. The suspension was filtered and washed to remove enzymes, and single cells and small capillary fragments were dispersed by triturating 4-7 times with a fire-polished glass Pasteur pipette. Cells were used within ˜6 hours after dispersion.

Arterial/arteriolar endothelial cell isolation. Single arterial/arteriolar endothelial cells (cECs) were obtained from mouse brains by first isolating arteries and arterioles, as previously described (Sonkusare et al., “Elementary Ca²⁺ signals through endothelial TRPV4 channels regulate vascular function,” Science 336(6081):597-601 (2012), which is hereby incorporated by reference in its entirety). Vessels were dissected in ice-cold artificial cerebrospinal fluid (composition previously explained). Arterial segments were transferred to dissociation solution composed of 55 mM NaCl, 80 mM Na-glutamate, 5.6 mM KCl, 2 mM MgCl₂, 4 mM glucose, and 10 mM HEPES (pH 7.3) containing neutral protease (0.5 mg/ml), elastase (0.5 mg/ml; Worthington, USA) and 100 μM CaCl₂, and incubated for 60 minutes at 37° C. Following this step, 0.5 mg/ml collagenase type I (Worthington, USA) was added and the solution was incubated for an additional 2 minutes at 37° C. The vessels were then mechanically disrupted to enhance endothelial cell liberation. Vascular fragments were washed to remove enzymes, and single endothelial cells were dispersed by triturating 5 times with a fire-polished glass Pasteur pipette. Cells were used within ˜6 hours after dispersion.

Arterial/arteriolar smooth muscle cell isolation. To isolate smooth muscle cells from intact cerebral arteries, vessel segments were placed in an isolation media (37° C., 10 minutes) containing 60 mM NaCl, 80 mM Na-glutamate, 5 mM KCl, 2 mM MgCl2, 10 mM glucose, and 10 mM HEPES with 1 mg/mL bovine serum albumin (BSA, pH 7.4). Arteries were then exposed to a 2-step digestion process that began with 14-minute incubation (37° C.) in media containing 0.5 mg/mL papain and 1.5 mg/mL dithioerythritol, followed by 10-minute incubation in media containing 100 μM Ca²⁺, 0.7 mg/mL type F collagenase, and 0.4 mg/mL type H collagenase. After incubation, tissues were washed repeatedly with ice-cold isolation media and triturated with a fire-polished pipette. Liberated cells were stored on ice for use on the same day.

Electrophysiology. Whole-cell currents were recorded using a patch-clamp amplifier (Axopatch 200B; Molecular Devices), filtered at 1 kHz, digitized at 5 kHz, and stored on a computer for offline analysis with Clampfit 10.3 software. Whole-cell capacitance was measured using the cancellation circuitry in the voltage-clamp amplifier. Electrophysiological analyses were performed in either the conventional or perforated whole-cell configuration. Recording pipettes were fabricated by pulling borosilicate glass (1.5 mm outer diameter, 1.17 mm inner diameter; Sutter Instruments, USA) using a Narishige puller. Pipettes were fire-polished to a tip resistance of ˜4-6 MΩ. The bath solution consisted of 80 mM NaCl, 60 mM KCl, 1 mM MgCl₂, 10 mM HEPES, 4 mM glucose, and 2 mM CaCl₂ (pH 7.4). For the conventional whole-cell configuration, pipettes were backfilled with a solution consisting of 10 mM NaOH, 11.4 mM KOH, 128.6 mM KCl, 1.1 mM MgCl₂, 2.2 mM CaCl₂, 5 mM EGTA, and 10 mM HEPES (pH 7.2). As noted in the Examples infra, the pipette solution was supplemented in some experiments with ATP (1 mM) or a derivative of PIP₂. For perforated-patch electrophysiology, the pipette solution was composed of 10 mM NaCl, 26.6 mM KCl, 110 mM K⁺ aspartate, 1 mM MgCl₂, 10 mM HEPES and 200-250 μg/ml amphotericin B, added freshly on the day of the experiment.

Ex vivo capillary-parenchymal arteriole (CaPA) preparation. The CaPA preparation was obtained by dissecting intracerebral arterioles arising from the M1 region of the middle cerebral artery, leaving the attached capillary bed intact. Precapillary arteriolar segments were cannulated on glass micropipettes with one end occluded by a tie and pressurized using a Living Systems Instrumentation (USA) pressure servo controller with mini peristaltic pump. The ends of the capillaries were then sealed by the downward pressure of an overlying glass micropipette. CaPA preparations were superfused (4 mL/min) with prewarmed (36° C.±1° C.), gassed (5% CO₂, 20% O₂, 75% N₂) artificial cerebrospinal fluid (aCSF) for at least 30 minutes. The composition of aCSF was 125 mM NaCl, 3 mM KCl, 26 mM NaHCO₃, 1.25 mM NaH₂PO₄, 1 mM MgCl₂, 4 mM glucose, 2 mM CaCl₂, pH 7.3 (with aeration with 5% CO₂). Application of pressure (40 mmHg) to the cannulated parenchymal arteriole segment in this preparation pressurized the entire tree and induced myogenic tone in the arteriolar segment. Only viable CaPA preparations, defined as those that developed pressure-induced myogenic tone greater than 15%, were used in subsequent experiments. Endothelial function was tested by assessing the vasodilator response to NS309 (1 μM), an activator of endothelial SK and IK potassium channels. Drugs were applied by addition to the superfusate. With this preparation, 10 mM K⁺was applied onto capillaries by pressure ejection from a glass micropipette (tip diameter, ˜5 μm) attached to a Picospritzer III (Parker, USA) at −5 psi for 20 seconds. Luminal diameter in parenchymal arteriole was acquired in two regions at 15 Hz using a CCD camera and the edge-detection software IonWizard 6.2 (IonOptix, USA). Changes in arteriolar diameter were calculated from the average luminal diameter measured over the last 10 seconds of stimulation and were normalized to the maximum dilatory responses in 0 mM Ca²⁺ bath solution at the end of each experiment.

Measurement of functional hyperemia in vivo. Functional hyperemia induced by whisker stimulation was measured in the mouse somatosensory cortex using laser Doppler flowmetry, with some modifications on previously described procedures (Girouard et al., “Astrocytic Endfoot Ca²⁺ and BK Channels Determine Both Arteriolar Dilation and Constriction,” Proc. Nat'l. Acad. Sci. 107(8):3811-6 (2010); Longden et al, “Capillary K⁺-Sensing Initiates Retrograde Hyperpolarization to Increase Local Cerebral Blood Flow,” Nat. Neurosci. 20:717-726 (2017), which are hereby incorporated by reference in their entirety). Briefly, animals were first anesthetized with isoflurane (5% induction, 2% maintenance) during the surgical procedure. A catheter was inserted into the femoral artery for monitoring blood pressure and collecting blood samples for blood gas analysis. A 2×2 mm cranial window was made over the somatosensory cortex after the head was immobilized on a custom-made stereotactic frame, and the dura was slit opened to allow a drug to access to the brain parenchyma. The site of cranial window was superfused with artificial cerebrospinal fluid (aCSF; 125 mM NaCl, 3 mM KCl, 26 mM NaHCO₃, 1.25 mM NaH₂PO₄, 2 mM CaCl₂, 1 mM MgCl₂ and 4 mM glucose, pH 7.3, ˜37° C.). Then, the anesthesia was switched to α-chloralose (50 mg/kg, i.p.) and urethane (750 mg/kg, i.p.) to avoid the effect of isoflurane, known as a strong vasodilator, on blood pressure and cerebral blood flow (CBF). Cortical CBF was recorded by laser Doppler probe (PeriMed) placed over the somatosensory cortex at the site distant from visible pial vessels through the cranial window. As CBF is expressed as an arbitrary unit, functional hyperemia response was measured as the percent change in CBF, induced by stroking the contralateral vibrissae at a frequency of ˜3 Hz for 1 min (i.e. whisker stimulation), from a baseline value. Pharmacological agents were topically applied by adding to the cortical superfusate with the exception of diC¹⁶—PIP₂ which was systemically administrated via the catheter inserted into the femoral artery. During CBF measurement, blood pressure was continuously recorded via a femoral artery cannula and body temperature was maintained at 37° C. by a servo-controlled heating pad with a rectal temperature sensor probe. The depth of anesthesia was assessed by monitoring blood pressure and reflex responses to tail pinch. All data were recorded and analyzed using LabChart software (AD instrument).

### Example 6—Inherent Barium-Sensitive Component of Functional Hyperemia is Absent in CADASIL Mouse Model but is Restored by HB-EGF Treatment

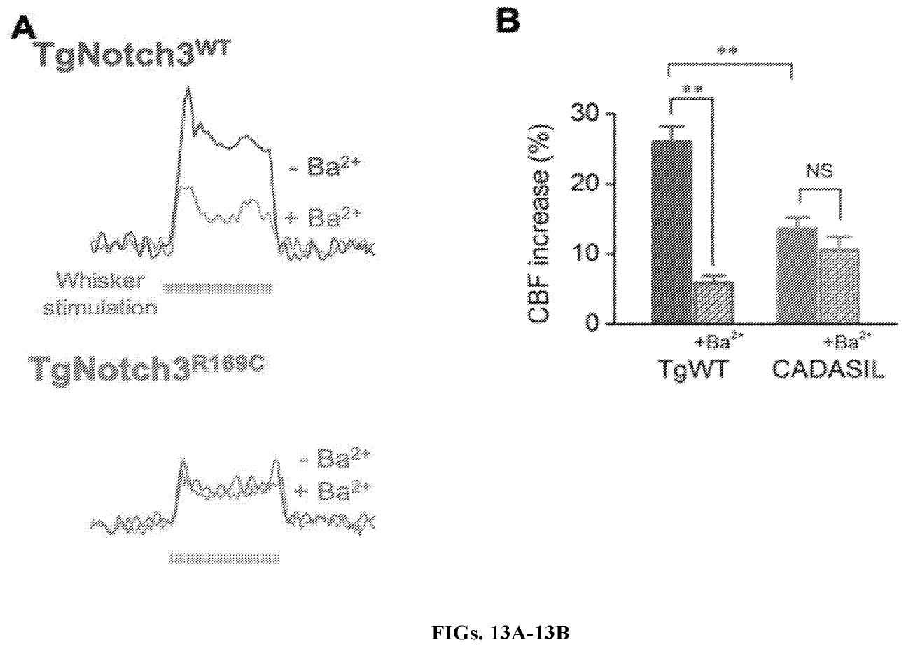

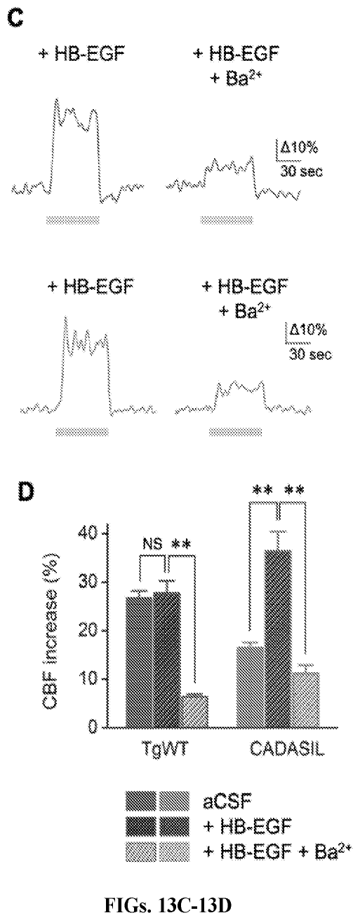

To investigate the effects of NOTCH3(R169C) expression on neurovascular coupling, cerebral blood flow (CBF) responses evoked by whisker stimulation were measured in the somatosensory cortex through a cranial window using laser Doppler flowmetry. Transgenic mice overexpressing WT NOTCH3 (TgNotch3^{WT}) were used as control group. Whisker stimulation-evoked CBF increases were markedly blunted in 6-mo-old TgNotch3^{R169C} mice compared to TgNotch3^{WT} mice, as previously reported (Joutel et al., “Cerebrovascular Dysfunction and Microcirculation Rarefaction Precede White Matter Lesions in a Mouse Genetic Model of Cerebral Ischemic Small Vessel Disease,” JCI 120:433-435 (2010); Capone et al., “Mechanistic Insights into a TIMP3-Sensitive Pathway Constitutively Engaged in the Regulation of Cerebral Hemodynamics,” eLife 5:e17536 (2016), which are hereby incorporated by reference in their entirety) (FIG. 13A). In physiological conditions, functional hyperemia is severely reduced by application of 100 μM barium (Ba²⁺), a potent pore blocker of Kir2 channels (Longden et al., “Vascular Inward Rectifier K⁺ Channels as External K⁺ Sensors in the Control of Cerebral Blood Flow,” Microcirculation 22(3):183-96 (2015); Girouard et al., “Astrocytic Endfoot Ca²⁺ and BK Channels Determine Both Arteriolar Dilation and Constriction,” Proc. Nat'l. Acad. Sci. 107(8):3811-6 (2010); Longden et al, “Capillary K⁺-Sensing Initiates Retrograde Hyperpolarization to Increase Local Cerebral Blood Flow,” Nat. Neurosci. 20:717-726 (2017), which are hereby incorporated by reference in their entirety). This concentration of barium does not affect other types of potassium channels (Nelson et al., “Physiological Roles and Properties of Potassium Channels in Arterial Smooth Muscle,” AJP 268(4 Pt 1):C799-822 (1995), which are hereby incorporated by reference in their entirety) and does not affect neural activity in vivo (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety). The reduction of functional hyperemia by barium largely reflects blocking of Kir2.1 channel in cECs, thus preventing K⁺-sensing and subsequent retrograde electrical signaling that causes upstream arteriolar dilation (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety). Accordingly, Ba²⁺ significantly decreased functional hyperemia in TgNotch3^{WT} by 60% (FIG. 13B). However, Ba²⁺ had no effect on CBF responses recorded in TgNotch3^{R169C} animals, suggesting a lack of capillary-to-arteriole electrical signaling when the CADASIL-causing mutation is expressed. These experiments were then repeated in presence of HB-EGF. As previously reported, 20 nM HB-EGF perfused over the cranial window restored CBF responses to whisker stimulation in CADASIL model to control levels, while it had no significant effect in control mice (FIG. 13C) (Capone et al., “Mechanistic Insights into a TIMP3-Sensitive Pathway Constitutively Engaged in the Regulation of Cerebral Hemodynamics,” eLife 5:e17536 (2016), which is hereby incorporated by reference in its entirety). Importantly, it was found that HB-EGF-mediated increase in FH in CADASIL was inhibited by Ba²⁺, similarly to control conditions, suggesting that functional hyperemia is restored by rescuing K⁺-induced capillary-to-arteriole electrical signaling (FIG. 13D).

### Example 7—Raising K⁺ Around Capillaries Fails to Induce Hyperemia and Upstream Arteriolar Dilation in CADASIL

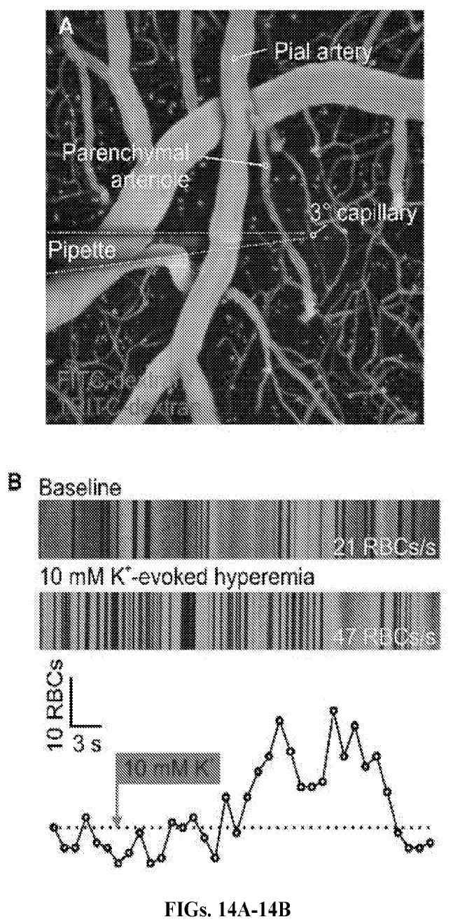

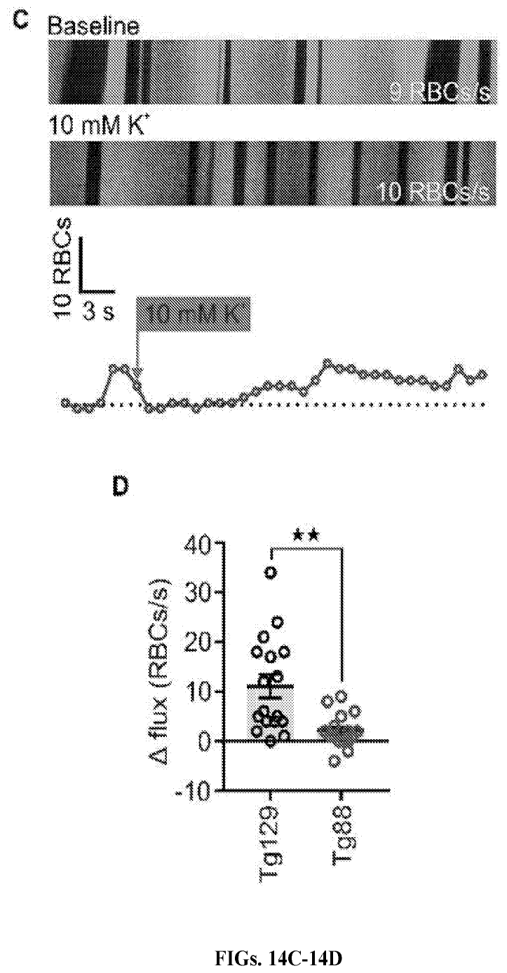

K⁺-induced upstream vasodilation in vivo was then tested by stimulating brain capillary with K⁺ and recorded red blood cell (RBC) flux through a cranial window using two-photon laser-scanning microscopy. Fluorescein isothiocyanate (FITC)-labeled dextran was injected in the circulation of anesthetized mice to visualize parenchymal microcirculation and enable RBC tracking (FIG. 14A). A pipette was positioned (tip diameter, 1-2 microns), containing artificial cerebrospinal fluid with 10 mM K⁺, adjacent to a capillary segment and raised local K⁺ by pressure ejection (5 PSI) for 300 ms. In control TgNotch3^{WT} mice, stimulus evoked a rapid increase in capillary RBC flux (Δ=11.1±2.3; n=17 animals). In contrast, elevation of external K⁺ had no effect on CADASIL (TgNotch3^{R169C}) mice (Δ=2.1±0.9; n=16 mice) (FIGS. 14B-14D).

Capillary hyperemia in response to K⁺ stimulus is caused by upstream arteriolar dilation and subsequent CBF increase (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety). To precisely track arteriolar diameter in response to focal capillary stimulation with K⁺, the innovative ex vivo capillary-parenchymal arteriole (CaPA) preparation was used (Longden et al, “Capillary K⁺-Sensing Initiates Retrograde Hyperpolarization to Increase Local Cerebral Blood Flow,” Nat. Neurosci. 20:717-726 (2017), which is hereby incorporated by reference in its entirety).

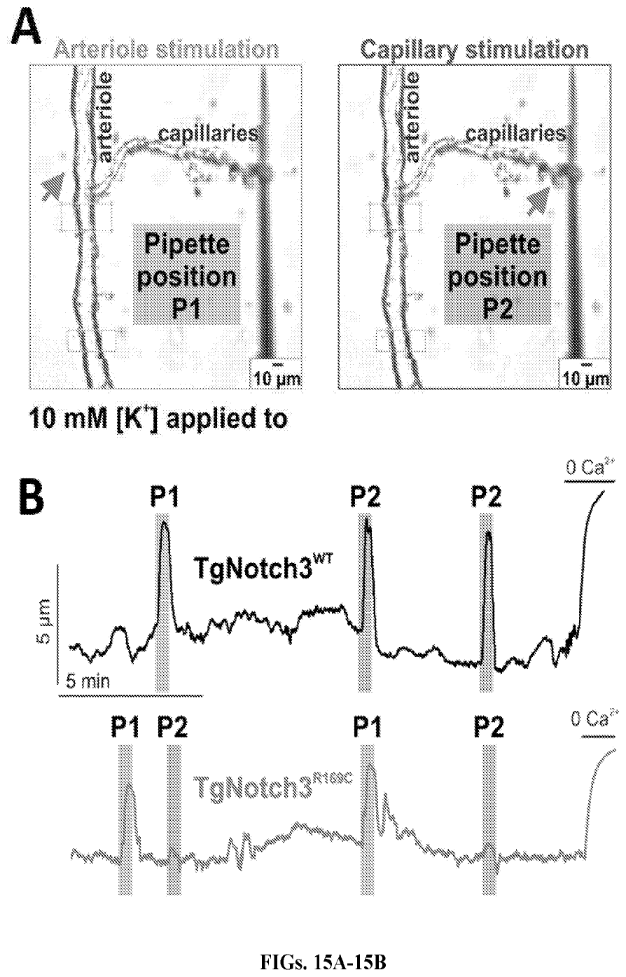

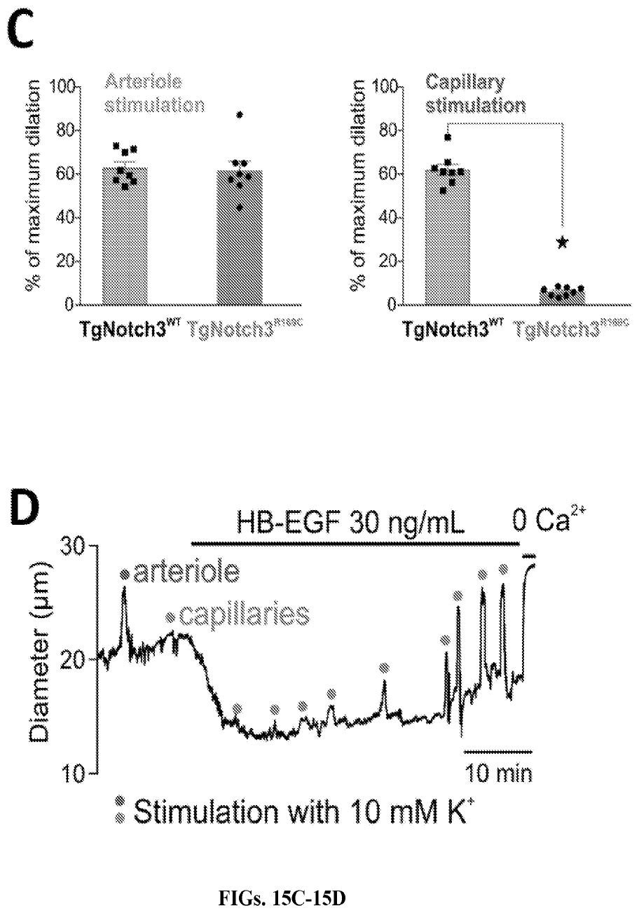

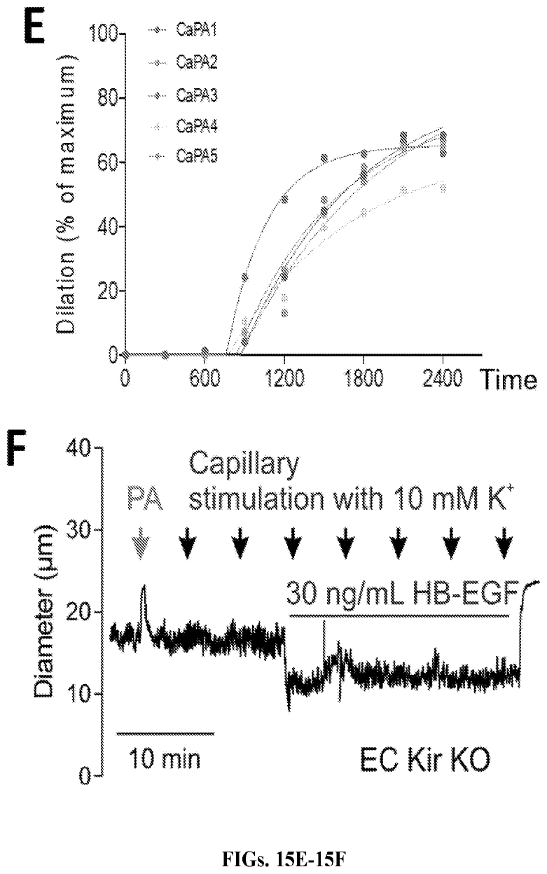

TABLE 1
Mean Values of Passive Diameter (Measured in the Absence of Extracellular
Ca²⁺), Active Diameter (After Development of Myogenic Tone), and Percentage of
Tone of the Arterioles used in FIG. 15.

| | Passive Diameter (μm) | | Active Diameter (μm) | | % Tone (40 mmHg) | |
|---|---|---|---|---|---|---|
| n | TgNotch3^{WT} | TgNotch3^{R169C} | TgNotch3^{WT} | TgNotch3^{R169C} | TgNotch3^{WT} | TgNotch3^{R169C} |
| 1 | 25.93 | 38.785 | 15.42 | 30.185 | 40.50 | 22.16 |
| 2 | 36.025 | 29.91 | 21.35 | 23.405 | 40.84 | 21.72 |
| 3 | 49.995 | 28.715 | 31.75 | 24.71 | 36.50 | 13.99 |
| 4 | 28.7 | 25.335 | 15.58 | 18.88 | 45.71 | 25.61 |
| 5 | 41.5105 | 41.16 | 24.32 | 34.07 | 41.67 | 17.25 |
| 6 | 25.345 | 36.185 | 16.02 | 27 08 | 36.82 | 25.12 |
| 7 | 21.38 | 30.34 | 14.04 | 23.9 | 34.36 | 21.32 |
| 8 | 16.49 | 34.695 | 9.48 | 24.98 | 42.45 | 27.67 |
| mean | 30.67 | 33.14 | 18.49 | 25.90 | 39.86 | 21.86 |
| s.e.m. | 3.92 | 1.92 | 2.47 | 1.63 | 1.31 | 1.59 |
| t-test | | 0.5842 | | 0.0277 | | 0.000001 |

Direct local stimulation of the arteriolar segment with 10 mM K⁺ by pressure ejection induced a reproducible dilatory response in CaPA preparations from both TgNotch3^{WT} and TgNotch3^{R169C} mice, showing similar vasodilatory abilities (FIGS. 15A-15C). However, when focal stimulus was applied on the capillary ends, arteriolar dilation was only observed in control condition, confirming a lack of capillary-to-arteriole signaling in the CADASIL model (FIGS. 15A-15C).

It was shown that CADASIL-causing mutation leads to a reduction in pressure-induced vasoconstriction (myogenic tone) of parenchymal arterioles and surface cerebral (pial) arteries (Dabertrand et al., “Potassium Channelopathy-like Defect Underlies Early-stage Cerebrovascular Dysfunction in a Genetic Model of Small Vessel Disease,” Proc. Nat'l. Acad. Sci. 112(7):E796-805 (2015), which is hereby incorporated by reference in its entirety). It was determined that the attenuation of myogenic tone is due an increase in the number of voltage gated K⁺ (Kv) channels in the cell membrane of arteriolar myocytes (Dabertrand et al., “Potassium Channelopathy-like Defect Underlies Early-stage Cerebrovascular Dysfunction in a Genetic Model of Small Vessel Disease,” Proc. Nat'l. Acad. Sci. 112(7):E796-805 (2015), which is hereby incorporated by reference in its entirety). The increase in Kv channel activity can be restored to normal by partial inhibition of Kv channels with 1 mM 4-aminopyridine (4-AP), and this restores myogenic tone. This maneuver did not restore arteriolar dilation in response to capillary stimulation with 10 mM K⁺ (FIG. 15C). The effect of 30 ng/mL HB-EGF was then tested which also restored myogenic tone presumably by promoting Kv1 channel endocytosis (Dabertrand et al., “Potassium Channelopathy-like Defect Underlies Early-stage Cerebrovascular Dysfunction in a Genetic Model of Small Vessel Disease,” Proc. Nat'l. Acad. Sci. 112(7):E796-805 (2015), which is hereby incorporated by reference in its entirety). Bath application of HB-EGF caused a rapid and sustained constriction in the arteriolar segment of CaPA prep from TgNotch3^{R169C} animals (FIG. 15D). Interestingly, after 17±0.5 minutes following restoration of the myogenic tone, arteriolar dilation in response to capillary stimulation with 10 mM K⁺ appeared and gradually increased to the amplitude observed in preparations from the control group (FIGS. 15D-15E). Finally, application of 10 mM K⁺ onto capillaries in presence of HB-EGF was without effect on upstream arteriole in CaPA preparations from endothelial specific Kir2.1^{-/-} mice, showing the necessary activation of capillary Kir2.1 channels to mediate HB-EGF effect (FIG. 15F).

### Example 8—Kir2-Mediated Currents are Decreased by 50% in Capillary Endothelial Cells from CADASIL but are Increased by HB-EGF

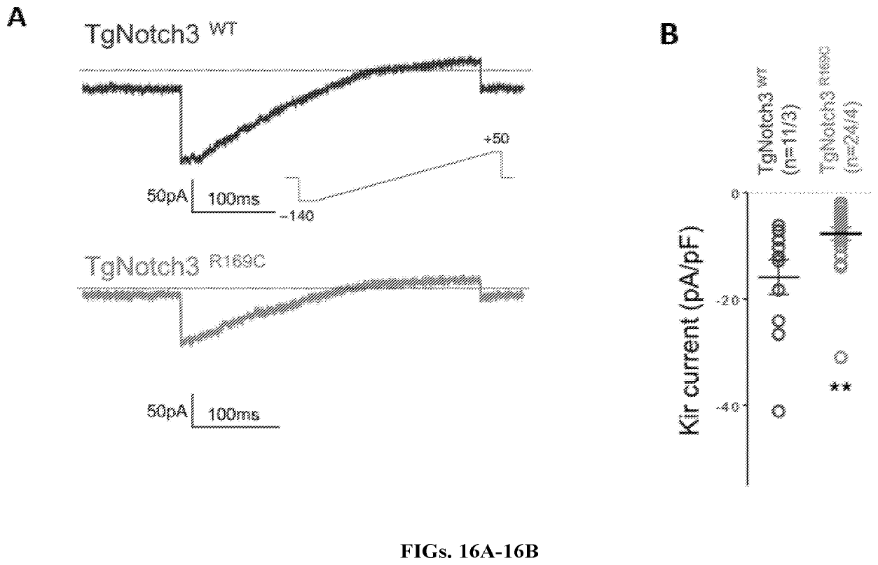

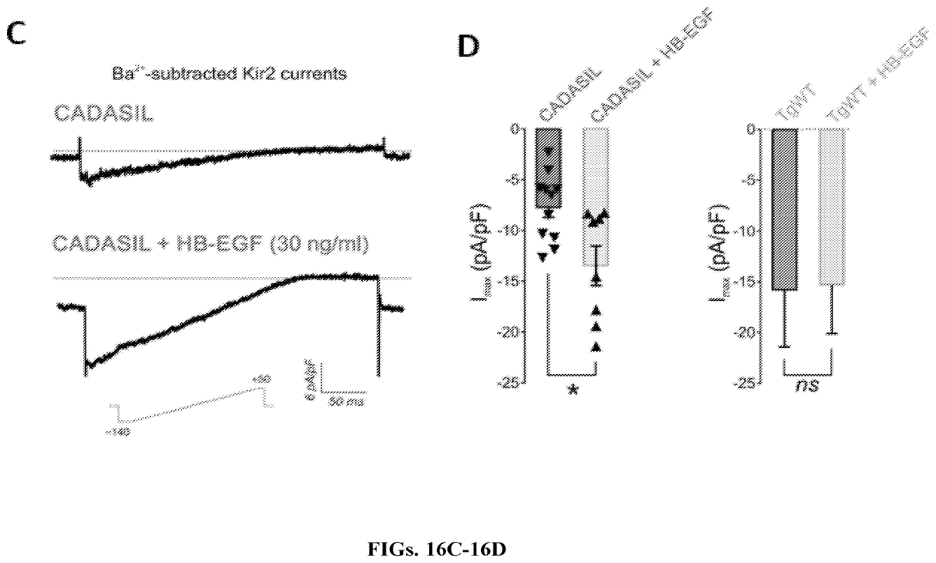

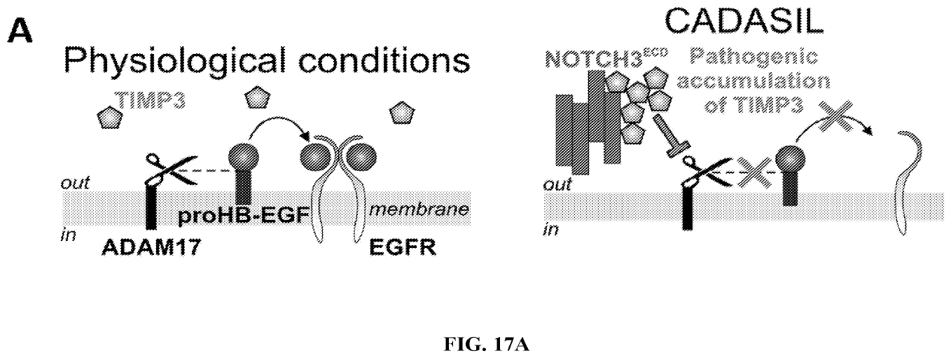

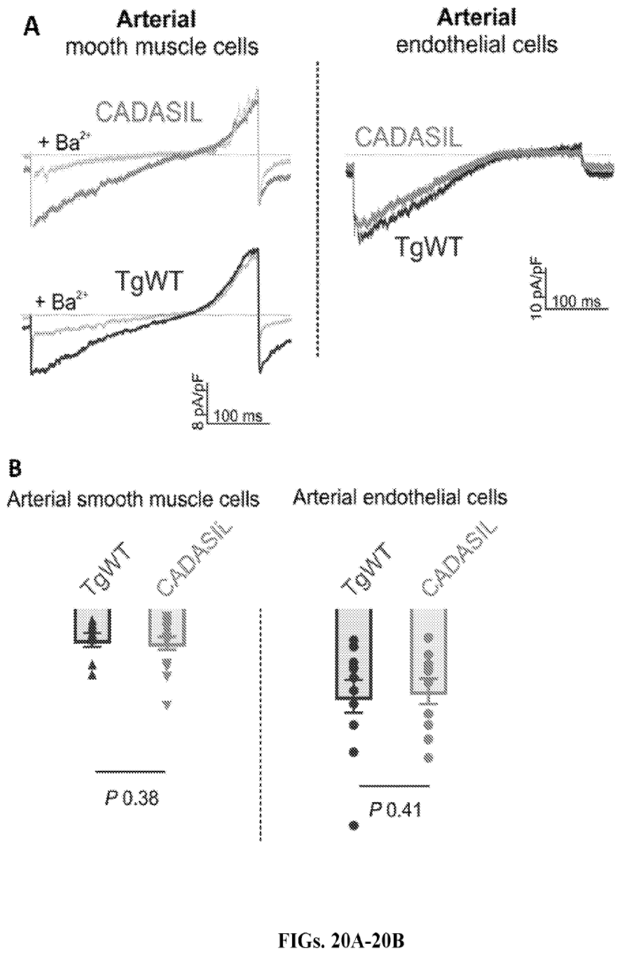

Because functional Kir2.1 channel in cECs is an absolute requirement for retrograde electrical signaling, Ba²⁺⁻ sensitive current density was investigated in freshly isolated capillary endothelial cells from TgNotch3^{WT} and TgNotch3^{R169C} brains. Currents were recorded in conventional whole cell configuration using 60 mM K⁺ bath solution to amplify Kir2.1 current amplitude. Patched cECs (holding potential −50 mV) were subjected to a 300-ms voltage-ramp from −140 to +50 mV, and the typical recorded current revealed a large ohmic inward component negative to K⁺ equilibrium potential EK (−23 mV at 60 mM K⁺), and a strongly rectifying component at potentials depolarized to EK. The inward component was sensitive to Ba²⁺, which was used to reveal the characteristic Kir2-current signature (FIG. 16A). CADASIL-causing mutation did not induce any measurable effect on Kir current densities from arteriolar endothelial and smooth muscle cells (FIGS. 20A-20B). However, current density appeared 50% lower in cECs from TgNotch3^{R169C} mice compared to control cECs (FIG. 16B). This is consistent with previous reports showing a ˜50% reduction in Kir2.1-current amplitude is sufficient to abolish capillary-to-arteriole electrical signaling. Furthermore, HB-EGF had no effect on current density from TgNotch3^{WT} cECs but restored it in cells from TgNotch3^{R169C} mice (FIGS. 16C-16D). Collectively, these results indicate that restoration of neurovascular coupling in CADASIL mouse by HB-EGF is accomplished by restoration of Kir2.1-mediated current in cECs.

### Example 9—Excess of TIMP3 Around Brain Capillary Endothelial Cells Blunts Kir2.1-Mediated Eectrical Signaling Through Inhibition of the ADAM17/HB-EGF/EGFR Module

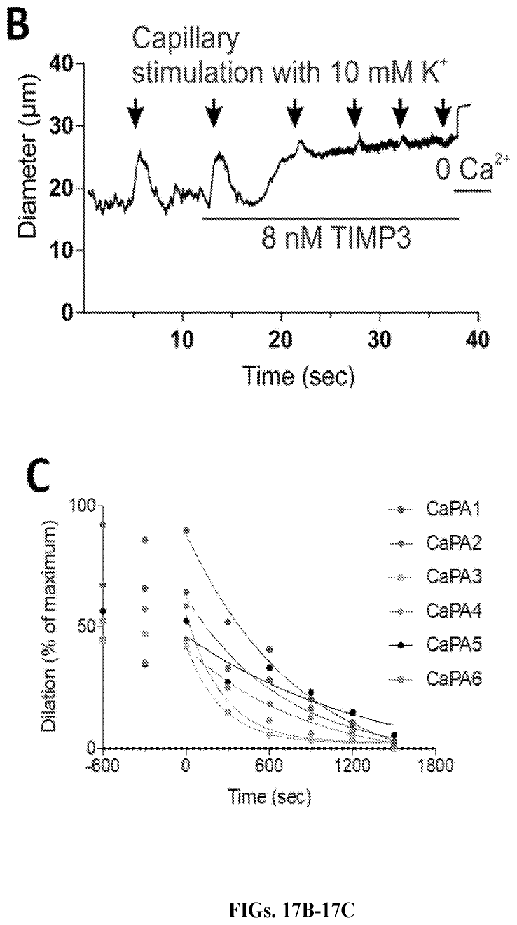

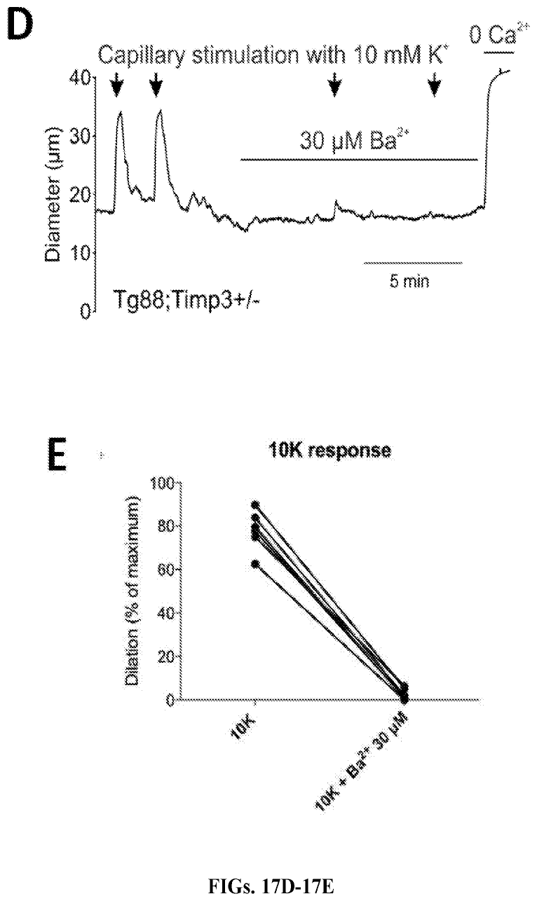

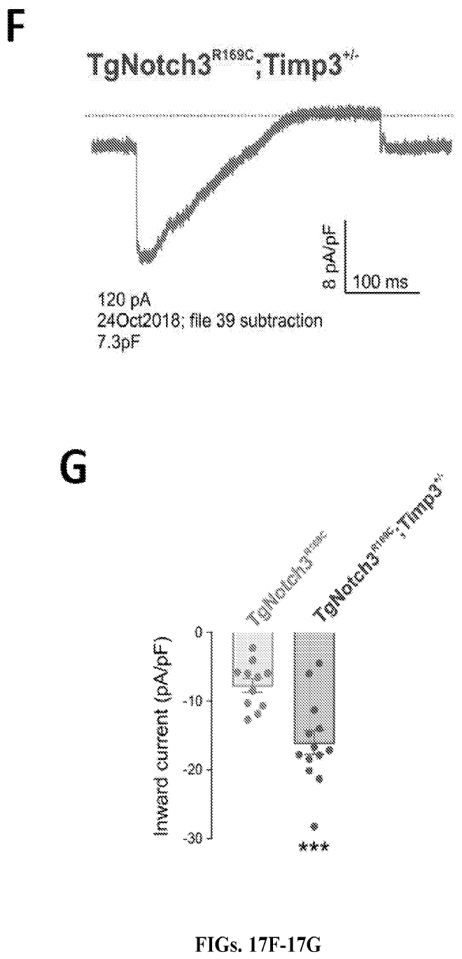

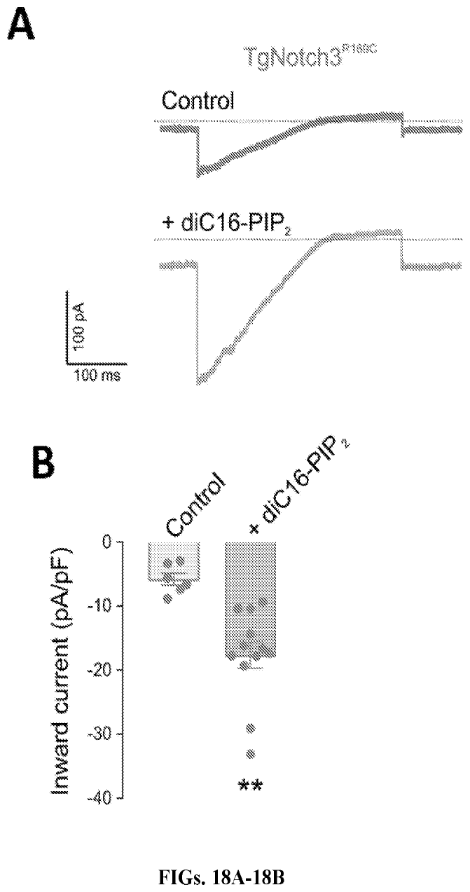

Perivascular accumulation of TIMP3 was previously identified as the pathological process leading to EGFR pathway inhibition and impaired cerebral hemodynamics in vivo (FIG. 17A) (Monet-Leprêtre et al., “Abnormal Recruitment of Extracellular Matrix Proteins by Excess Notch3 ECD: a New Pathomechanism in CADASIL,” Brain 136:1830-1845 (2013); Dabertrand et al., “Potassium Channelopathy-like Defect Underlies Early-stage Cerebrovascular Dysfunction in a Genetic Model of Small Vessel Disease,” Proc. Nat'l. Acad. Sci. 112(7):E796-805 (2015); Capone et al., “Reducing Timp3 or Vitronectin Ameliorates Disease Manifestations in CADASIL Mice,” Ann Neurol 79(3):387-403 (2016); Capone et al., “Mechanistic Insights into a TIMP3-Sensitive Pathway Constitutively Engaged in the Regulation of Cerebral Hemodynamics,” eLife 5:e17536 (2016), which are hereby incorporated by reference in their entirety). The effect of recombinant TIMP3 application on capillary-to-arteriole electrical signaling ex vivo was then investigated. In CaPA preparation from TgNotch3^{WT} animals, bath application of 8 nM soluble TIMP3 gradually attenuated and, ultimately, abolished arteriolar vasodilation induced by capillary stimulation with 10 mM K⁺ (FIGS. 17B-17C). This finding suggests that excess of TIMP3 impairs NVC responses in CADASIL by suppressing the ADAM17/HB-EGF/EGFR pathway at the capillary level (FIG. 17A). The contribution of TIMP3 accumulation to the CADASIL pathomechanism was then probed by genetically reducing Timp3 expression in a TgNotch3^{R169C}; Timp3^{+/-} double-mutant approach. Consistent with a previous report that Timp3 haploinsufficiency protects against attenuated functional hyperemia (Capone et al., “Mechanistic Insights into a TIMP3-Sensitive Pathway Constitutively Engaged in the Regulation of Cerebral Hemodynamics,” eLife 5:e17536 (2016), which is hereby incorporated by reference in its entirety), K⁺-induced upstream vasodilation appeared functional and completely abolished by Kir2 channel inhibitor Ba²⁺ in TgNotch3^{R169C}; Timp3^{+/-} mice (FIGS. 17D-17E). Finally, Kir2.1 currents were significantly higher in isolated cECs from TgNotch3^{R169C}; Timp3^{+/-} brains compared to TgNotch3^{R169C} brains (FIGS. 17F-17G).

### Example 10—Novel Therapeutic Approach Using Exogenous Phosphatidylinositol 4,5-Bisphosphate (PIP₂) to Restore Neurovascular Coupling in CADASIL Mouse Model

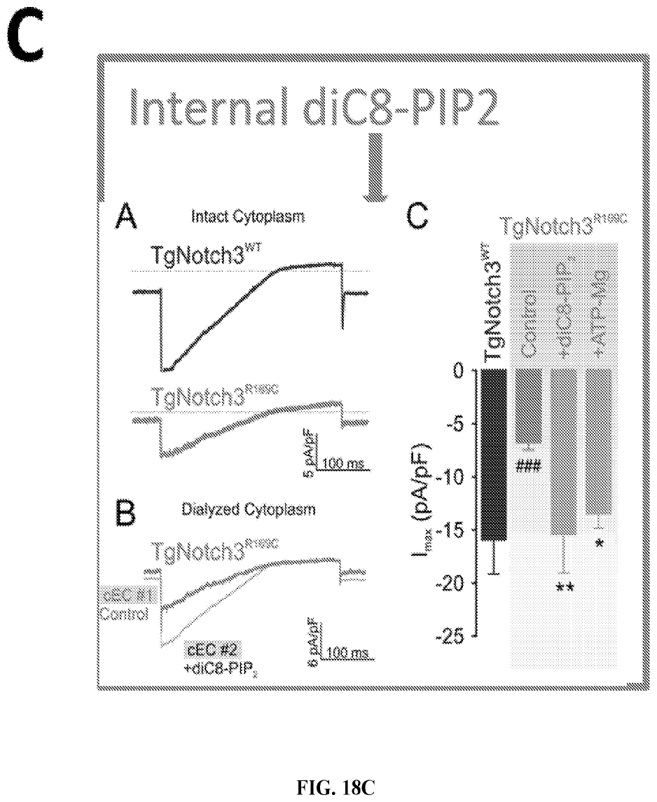

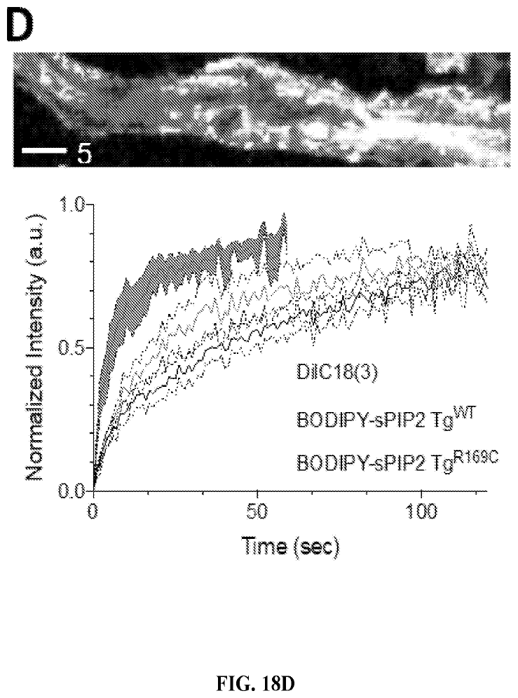

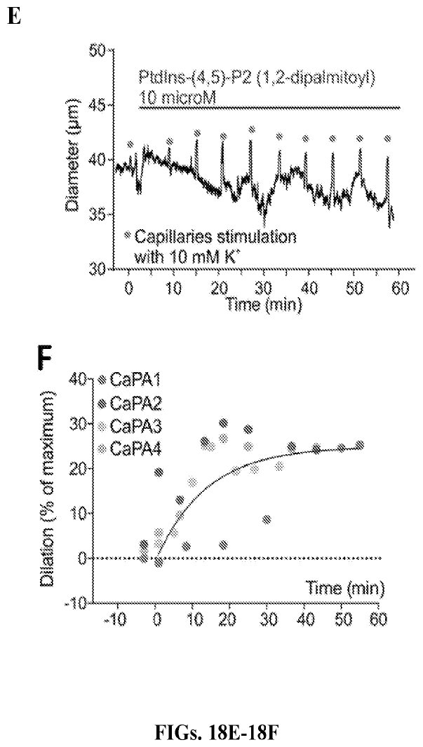

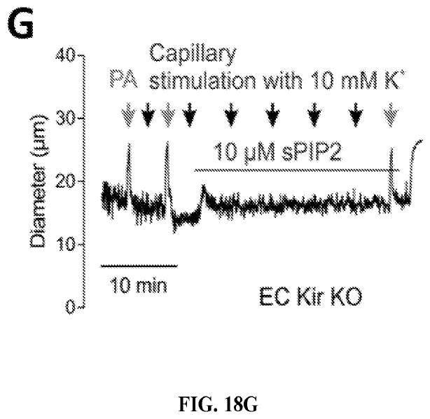

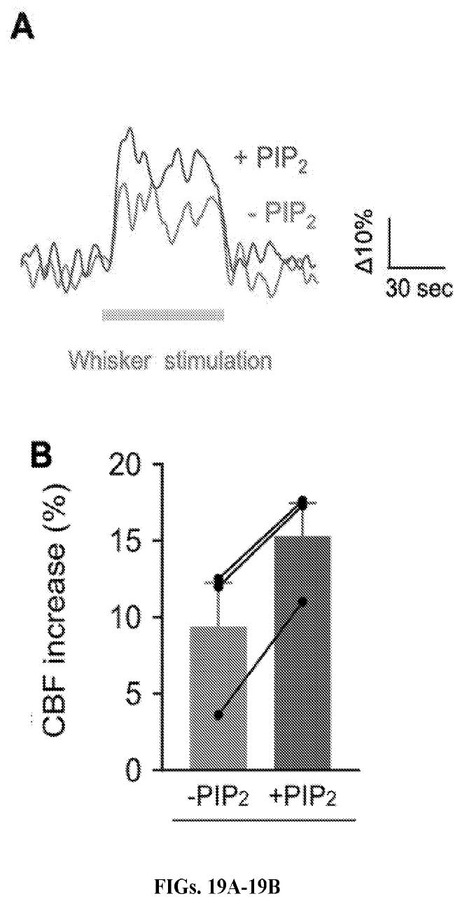

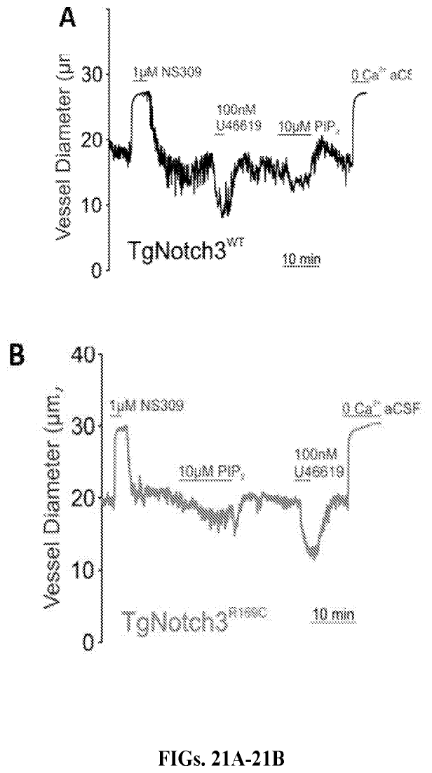

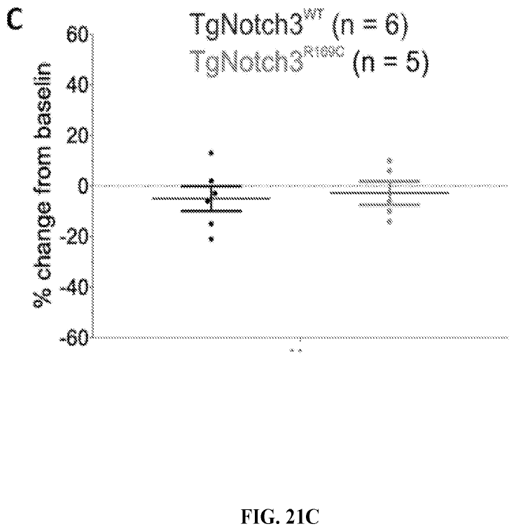

HB-EGF is a potent inducer of angiogenesis and cell growth, hence tumor progression, which limits its therapeutic potential. A novel potential therapeutic approach was developed based on an exogenous PIP₂ application since Kir2.1-mediated current is decreased by 50% in CADASIL. Exogenous application of soluble PIP₂ 10 μM increased Kir2-mediated current in cECs from CADASIL mice to values observed in control groups (FIGS. 18A-18B). Similarly, intracellular addition of soluble PIP₂ via the patch pipette counteracted the reduction in Kir current caused by the mutation (FIG. 18C). Fluorescence recovery after photobleaching (FRAP) was used to assess the mobility of exogenous PIP₂ labelled with a BODIPY fluorophore in the plasma membrane of cECs (FIG. 18D). Finally, addition of exogenous PIP₂ restored capillary-to-arteriole electrical signaling in CaPA prep ex vivo and functional hyperemia in vivo (FIGS. 18E-G and FIGS. 19A-19B). Eogenous PIP₂ has a negligible effect on isolated intracerebral arterioles diameter (FIGS. 21A-21C).

### Discussion of Examples 6-10

An invaluable tool in the efforts to advance the understanding of these diseases has been a well-characterized mouse model of CADASIL—the most common monogenic SVD—caused by stereotyped mutations in the extracellular domain (ECD) of the NOTCH3 receptor (NOTCH3^{ECD}). Using this mouse model, common defects have been discovered in the extracellular matrix (ECM) that cause early deficits in cerebral blood flow (CBF) control through alterations in the activity of microvascular ion channels. The ‘Holy Grail’ of this effort is to restore perfusion in an SVD setting and following ischemic stroke. Important in this context, it is possible to rapidly reverse functional hyperemia deficits in CADASIL model animals by normalizing elements of the comprised ECM pathway through exogenous addition or genetic correction, an accomplishment directly relevant to ischemic stroke. It has also been found that FH can be restored by supplying PIP₂ exogenously, an observation that holds significant therapeutic promise.

Although preferred embodiments have been depicted and described in detail herein, it will be apparent to those skilled in the relevant art that various modifications, additions, substitutions, and the like can be made without departing from the spirit of the present application and these are therefore considered to be within the scope of the present application as defined in the claims which follow.

What is claimed:

1. A method of treating a subject for a condition characterized by reduced cerebral blood flow, said method comprising:
selecting a subject having a condition characterized by reduced cerebral blood flow and
administering, to the selected subject, a therapeutic agent that increases the level of phosphatidylinositol 4,5-bisphosphate (PIP₂), under conditions effective to treat the condition characterized by reduced cerebral blood flow.

2. The method of claim 1, wherein the therapeutic agent is a small molecule.

3. The method of claim 1, wherein the therapeutic agent is a soluble PIP₂ analog.

4. The method of claim 3, wherein the soluble PIP₂ analog is selected from the group consisting of diC4-PIP₂, diC6-PIP₂, diC8-PIP₂ (08:0 PIP2), diC16-PIP₂, diC18:1 PIP₂, 18:0-20:4 PIP₂2, and brain PIP₂.

5. The method of claim 1, wherein the therapeutic agent is selected from the group consisting of edelfosine, miltefosine, perifosine, erucylphosphocholine, alkylphosphocholine, ilmofosine, BN 52205, BN 5221.1, 2-fluoro-3-hexadecyloxy-2-methylprop-1-yl 2′-(trimethylammonio) ethyl phosphate, and LY294002.

6. The method of claim 1, wherein the condition characterized by reduced cerebral blood flow is selected from the group consisting of a small vessel disease, ischemic stroke, traumatic brain injury, and cerebral ischemia.

7. The method of claim 6, wherein the condition characterized by reduced cerebral blood flow is a small vessel disease.

8. The method of claim 7, wherein the small vessel disease comprises cerebral autosomal-dominant arteriopathy with subcortical infarcts and leukoencephalopathy (CADASIL).

9. The method of claim 1, wherein said administering is performed orally, topically, transdermally, parenterally, intradermally, intracisternally, intramuscularly, intraperitoneally, intravenously, subcutaneously, by intranasal instillation, by intracavitary or intravesical instillation, intraocularly, intraarterially, intralesionally, by application to mucous membranes, by catheterization, implantation, direct injection, dermal/transdermal application, or portal vein administration to relevant tissues.

10. A method of treating cerebral autosomal-dominant arteriopathy with subcortical infarcts and leukoencephalopathy (CADASIL) in a subject, said method comprising:
selecting a subject having cerebral autosomal-dominant arteriopathy with subcortical infarcts and leukoencephalopathy (CADASIL) and
administering, to the selected subject, a therapeutic agent that increases the level of phosphatidylinositol 4,5-bisphosphate (PIP₂), under conditions effective to treat CADASIL in the selected subject.

11. The method of claim 10, wherein the therapeutic agent is a small molecule.

12. The method of claim 10, wherein the therapeutic agent is a soluble PIP₂ analog.

13. The method of claim 12, wherein the soluble PIP₂ analog is selected from the group consisting of diC4-PIP₂, diC6-PIP₂, diC8-PIP₂ (08:0 PIP₂), diC16-PIP₂, diC18:1 PIP₂, 18:0-20:4 PIP₂, and brain PIP₂.

14. The method of claim 10, wherein the therapeutic agent is selected from the group consisting of edelfosine, miltefosine, perifosine, erucylphosphocholine, alkylphosphocholine, ilmofosine, BN 52205, BN 5221.1, 2-fluoro-3-hexadecyloxy-2-methylprop-1-yl 2′-(trimethylammonio) ethyl phosphate, and LY294002.

15. The method of claim 10, wherein said administering is performed orally, topically, transdermally, parenterally, intradermally, intracisternally, intramuscularly, intraperitoneally, intravenously, subcutaneously, by intranasal instillation, by intracavitary or intravesical instillation, intraocularly, intraarterially, intralesionally, by application to mucous membranes, by catheterization, implantation, direct injection, dermal/transdermal application, or portal vein administration to relevant tissues.

16. A method of restoring cerebral blood flow in a subject, said method comprising:
selecting a subject having a reduction in cerebral blood flow and
administering, to the selected subject, a therapeutic agent that increases the level of phosphatidylinositol 4,5-bisphosphate (PIP₂), under conditions effective to restore cerebral blood flow in the selected subject.

17. The method of claim 16, wherein the therapeutic agent is a small molecule.

18. The method of claim 16, wherein the therapeutic agent is a soluble PIP₂ analog.

19. The method of claim 18, wherein the soluble PIP₂ analog is selected from the group consisting of diC4-PIP₂, diC6-PIP₂, diC8-PIP₂ (08:0 PIP₂), diC16-PIP₂, diC18:1 PIP₂, 18:0-20:4 PIP₂, and brain PIP₂.

20. The method of claim 16, wherein the therapeutic agent is selected from the group consisting of edelfosine, miltefosine, perifosine, erucylphosphocholine, alkylphosphocholine, ilmofosine, BN 52205, BN 5221.1, 2-fluoro-3-hexadecyloxy-2-methylprop-1-yl 2′-(trimethylammonio) ethyl phosphate, and LY294002.

21. The method of claim 16, wherein said subject has a condition characterized by reduced cerebral blood flow.

22. The method of claim 16, wherein said administering is performed orally, topically, transdermally, parenterally, intradermally, intracisternally, intramuscularly, intraperitoneally, intravenously, subcutaneously, by intranasal instillation, by intracavitary or intravesical instillation, intraocularly, intraarterially, intralesionally, by application to mucous membranes, by catheterization, implantation, direct injection, dermal/transdermal application, or portal vein administration to relevant tissues.

23. A method of restoring functional hyperemia in a subject, said method comprising:
selecting a subject having reduced functional hyperemia and
administering, to the selected subject, a therapeutic agent that increases the level of phosphatidylinositol 4,5-bisphosphate (PIP₂), under conditions effective to restore functional hyperemia, in the selected subject.

24. The method of claim 23, wherein the therapeutic agent is a small molecule.

25. The method of claim 23, wherein the therapeutic agent is a soluble PIP₂ analog.

26. The method of claim 25, wherein the soluble PIP₂ analog is selected from the group consisting of diC4-PIP₂, diC6-PIP₂, diC8-PIP₂ (08:0 PIP₂), diC16-PIP₂, diC18:1 PIP₂, 18:0-20:4 PIP₂, and brain PIP₂.

27. The method of claim 23, wherein the therapeutic agent is selected from the group consisting of edelfosine, miltefosine, perifosine, erucylphosphocholine, alkylphosphocholine, ilmofosine, BN 52205, BN 5221.1, 2-fluoro-3-hexadecyloxy-2-methylprop-1-yl 2′-(trimethylammonio) ethyl phosphate, and LY294002.

28. The method of claim 23, wherein said subject has a condition characterized by reduced functional hyperemia.

29. The method of claim 23, wherein said administering is performed orally, topically, transdermally, parenterally, intradermally, intracisternally, intramuscularly, intraperitoneally, intravenously, subcutaneously, by intranasal instillation, by intracavitary or intravesical instillation, intraocularly, intraarterially, intralesionally, by application to mucous membranes, by catheterization, implantation, direct injection, dermal/transdermal application, or portal vein administration to relevant tissues.
